(19) United States
(12) Patent Application Publication
(10) Pub. No.: US 2021/0257051 A1
(43) Pub. Date: Aug. 19, 2021

(54) A METHOD FOR ANALYSIS OF REAL-TIME AMPLIFICATION DATA

(71) Applicant: IMPERIAL COLLEGE OF SCIENCE, TECHNOLOGY AND MEDICINE, London (GB)

(72) Inventors: Pantelis GEORGIOU, London (GB); Ahmad MONIRI, London (GB); Jesus RODRIGUEZ-MANZANO, London (GB)

(73) Assignee: IMPERIAL COLLEGE OF SCIENCE, TECHNOLOGY AND MEDICINE, London (GB)

(21) Appl. No.: 16/973,410

(22) PCT Filed: Jun. 7, 2019
(86) PCT No.: PCT/EP2019/065039

§ 371 Date: Dec. 8, 2020

(30) Foreign Application Priority Data
Jun. 8, 2018 (GB) 1809418.5

(51) Int. Cl. G16B 25/20; C12Q 1/6851

(52) U.S. Cl. CPC G16B 25/20; C12Q 1/6851

(57) ABSTRACT

This disclosure relates to methods, systems, computer programs and computer-readable media for the multidimensional analysis of real-time amplification data. A framework is presented that shows that the benefits of standard curves extend beyond absolute quantification when observed in a multidimensional environment. Relating to the field of Machine Learning, the disclosed method combines multiple extracted features (e.g. linear features) in order to analyse real-time amplification data using a multidimensional view. The method involves two new concepts: the multidimensional standard curve and its ‘home’, the feature space. Together they expand the capabilities of standard curves, allowing for simultaneous absolute quantification, outlier detection and providing insights into amplification kinetics. The new methodology thus enables enhanced quantification of nucleic acids, single-channel multiplexing, outlier detection, characteristic patterns in the multidimensional space related to amplification kinetics and increased robustness for sample identification and quantification.

### RELATED APPLICATIONS

The present application is a National Phase entry of PCT Application No. PCT/EP2019/065039, filed Jun. 7, 2019, which claims priority from Great Britain Application No. 1809418.5 filed Jun. 8, 2018, all of these disclosures being hereby incorporated by reference in their entirety.

### TECHNICAL FIELD

This disclosure relates to methods, systems, computer programs and computer-readable media for the multidimensional analysis of real-time amplification data.

### BACKGROUND

Since its inception, the real-time polymerase chain reaction (qPCR) has become a routine technique in molecular biology for detecting and quantifying nucleic acids. This is predominantly due to its large dynamic range (7-8 orders of magnitude), desirable sensitivity (5-10 molecules) and reproducible quantification results. New methods to improve the analysis of qPCR data are invaluable to a number of analytical fields, including environmental monitoring and clinical diagnostics. Absolute quantification of nucleic acids in real-time PCR using standard curves is undoubtedly important and significant in various fields of biomedicine, although research has saturated in recent years.

The current “gold standard” for absolute quantification of a specific target sequence is the cycle-threshold (Ct) method. The Cₜ value is a feature of the amplification curve defined as the number of cycles in the exponential region where there is a detectable increase in fluorescence. Since this method has been proposed, several alternative methods have been developed in a hope to improve absolute quantification in terms of accuracy, precision and robustness. The focus of existing research has been based on the computation of single features, such as C_{y} and −log₁₀(F₀), that are linearly related to initial concentration. This provides a simple approach for absolute quantification, however, data analysis based on such single features has been limited. Thus, research into improving methods for absolute quantification of nucleic acids using standard curves has plateaued and is very incremental in improvement.

Rutledge et al. 2004 proposed the Sigmoidal curve-fitting (SCF) for quantification based on three kinetic parameters (Fc, Fₘₐₓ and F₀). Sisti et al. 2010 developed the “shape-based outlier detection” method, which is not based on amplification efficiency and uses a non-linear fitting to parameterize PCR amplification profiles. The shape-based outlier detection method takes a multidimensional approach in order to define a similarity measure between amplification curves, but relies on using a specific model for amplification, namely the 5-parameter sigmoid, and is not a general method. Furthermore, the shape-based outlier detection method is typically used as an add-on, and only uses a multidimensional approach for outlier detection, such that quantification is only considered using a unidimensional approach. Guescini et al. 2013 proposed the C_{y0} method, which is similar to the Ct method but takes into account the kinetic parameters of the amplification curve and may compensate for small variations among the samples being compared. Bar et al. 2013 proposed a method (KOD) based on amplification efficiency calculation for the early detection of non-optimal assay conditions.

The present disclosure aims to at least partially overcome the problems inherent in existing techniques.

### SUMMARY

The invention is defined by the appended claims. The supporting disclosure herein presents a framework that shows that the benefits of standard curves extend beyond absolute quantification when observed in a multidimensional environment. The focus of existing research has been on the computation of a single value, referred to herein as a “feature”, that is linearly related to target concentration, and thus there has been a gap in existing approaches in terms of taking advantage of multiple features. It has now been realised that the benefits of combining linear features are non-trivial. Previous methods have been restricted to the simplicity of conventional standard curves such as the gold standard cycle-threshold (Ct) method. This new methodology enables enhanced quantification of nucleic acids, single-channel multiplexing, outlier detection, characteristic patterns in the multidimensional space related to amplification kinetics and increased robustness for sample identification and quantification.

Relating to the field of Machine Learning, the presently disclosed method takes a multidimensional view, combining multiple features (e.g. linear features) in order to take advantage of, and improve on, information and principles behind existing methods to analyze real-time amplification data. The disclosed method involves two new concepts: the multidimensional standard curve and its ‘home’, the feature space. Together they expand the capabilities of standard curves, allowing for simultaneous absolute quantification, outlier detection and providing insights into amplification kinetics. This disclosure describes a general method which, for the first time, presents a multi-dimensional standard curve, increasing the degrees of freedom in data analysis and thereby being capable of uncovering trends and patterns in real-time amplification data obtained by existing qPCR instruments (such as the LightCycler 96 System from Roche Life Science). It is believed that this disclosure redefines the foundations of analysing real-time nucleic acid amplification data and enables new applications in the field of nucleic acid research.

In a first aspect of the disclosure there is provided a method for use in quantifying a sample comprising a target nucleic acid, the method comprising: obtaining a set of first real-time amplification data for each of a plurality of target concentrations; extracting a plurality of N features from the set of first data, wherein each feature relates the set of first data to the concentration of the target; and fitting a line to a plurality of points defined in an N-dimensional space by the features, each point relating to one of the plurality of target concentrations, wherein the line defines a multidimensional standard curve specific to the nucleic acid target which can be used for quantification of target concentration.

Optionally the method further comprises: obtaining second real-time amplification data relating to an unknown sample; extracting a corresponding plurality of N features from the second data; and calculating a distance measure between the line in N-dimensional space and a point defined in N-dimensional space by the corresponding plurality of N features. Optionally, the method further comprises computing a similarity measure between amplification curves from the distance measure, which can optionally be used to identify outliers or classify targets.

Optionally each feature is different to each of the other features, and optionally wherein each feature is linearly related to the concentration of the target, and optionally wherein one or more of the features comprises one of Cₜ, C_{y} and −log₁₀(F₀).

Optionally the method further comprises mapping the line in N-dimensional space to a unidimensional function, M₀, which is related to target concentration, and optionally wherein the unidimensional function is linearly related to target concentration, and/or optionally wherein the unidimensional function defines a standard curve for quantifying target concentration. Optionally, the mapping is performed using a dimensionality reduction technique, and optionally wherein the dimensionality reduction technique comprises at least one of: principal component analysis; random sample consensus; partial-least squares regression; and projecting onto a single feature. Optionally, the mapping comprises applying a respective scalar feature weight to each of the features, and optionally wherein the respective feature weights are determined by an optimization algorithm which optimizes an objective function, and optionally wherein the objective function is arranged for optimization of quantisation performance.

Optionally, calculating the distance measure comprises projecting the point in N-dimensional space onto a plane which is normal to the line in N-dimensional space, and optionally wherein calculating the distance measure further comprises calculating, based on the projected point, a Euclidean distance and/or a Mahalanobis distance. Optionally, the method further comprises calculating a similarity measure based on the distance measure, and optionally wherein calculating a similarity measure comprises applying a threshold to the similarity measure. Optionally, the method further comprises determining whether the point in N-dimensional space is an inlier or an outlier based on the similarity measure. Optionally, the method further comprises: if the point in N-dimensional space is determined to be an outlier then excluding the point from training data upon which the step of fitting a line to a plurality of points defined in N-dimensional space is based, and if the point in N-dimensional space is not determined to be an outlier then re-fitting the line in N-dimensional space based additionally on the point in N-dimensional space.

Optionally, the method further comprises determining a target concentration based on the multidimensional standard curve, and optionally further based on the distance measure, and optionally when dependent on claim 4 based on the unidimensional function which defines the standard curve. Optionally, the method further includes displaying the target concentration on a display.

Optionally, the method further comprises a step of fitting a curve to the set of first data, wherein the feature extraction is based on the curve-fitted first data, and optionally wherein the curve fitting is performed using one or more of a 5-parameter sigmoid, an exponential model, and linear interpolation. Optionally, the set of first data relating to the melting temperatures is pre-processed, and the curve fitting is carried out on the processed set of first data, and optionally wherein the pre-processing comprises one or more of: subtracting a baseline; and normalization.

Optionally, the data relating to the melting temperature is derived from one or more physical measurements taken versus sample temperature, and optionally wherein the one or more physical measurements comprise fluorescence readings.

In a second aspect there is provided a system comprising at least one processor and/or at least one integrated circuit, the system arranged to carry out a method according to the first aspect.

In a third aspect there is provided a computer program comprising instructions which, when executed by one or more processors, cause the one or more processors to perform a method according to the first aspect.

In a fourth aspect there is provided a computer-readable medium storing instructions which when executed by at least one processor, cause the at least one processor to carry out a method according to the first aspect.

In a fifth aspect there is provided a method according to the first aspect, used for detection of genomic material, and optionally wherein the genomic material comprises one or more pathogens, and optionally wherein the pathogens comprise one more carbapenemase-producing enterobacteria, and optionally wherein the pathogens comprise one or more carbapenemase genes from the set comprising blaOXA-48, blaVIM, blaNDM and blaKPC.

In a sixth aspect there is provided a method for diagnosis of an infection by detection of one or more pathogens according to the method of the first aspect, and optionally wherein the pathogens comprise one more carbapenemase-producing enterobacteria, and optionally wherein the pathogens comprise one or more carbapenemase genes from the set comprising blaOXA-48, blaVIM, blaNDM and blaKPC.

In a seventh aspect there is provided a method for point-of-care diagnosis of an infectious disease by detection of one or more pathogens according to the method of the first aspect, and optionally wherein the pathogens comprise one more carbapenemase-producing enterobacteria, and optionally wherein the pathogens comprise one or more carbapenemase genes from the set comprising blaOXA-48, blaVIM, blaNDM and blaKPC.

The methods disclosed herein, if used for diagnosis, can be performed in vitro or ex vivo. Embodiments can be used for single-channel multiplexing without post-PCR manipulations.

It will be appreciated in the light of the present disclosure that certain features of certain aspects and/or embodiments described herein can be advantageously combined with those of other aspects and/or embodiments. The following description of specific embodiments should not therefore be interpreted as indicating that all of the described steps and/or features are essential. Instead, it will be understood that certain steps and/or features are optional by virtue of their function or purpose, even where those steps or features are not explicitly described as being optional. The above aspects are thus not intended to limit the invention, and instead the invention is defined by the appended claims.

### BRIEF DESCRIPTION OF THE FIGURES

In order that the disclosure may be understood, preferred embodiments are described below, by way of example, with reference to the Figures in which like features are provided with like reference numerals. Figures are not necessarily drawn to scale.

FIG. 1 is a representation of training and testing in an existing unidimensional approach, compared with the proposed multidimensional framework.

FIGS. 2a-2c illustrate the process of training using the multidimensional approach described herein.

FIGS. 2d-2f illustrate the process of testing using the multidimensional approach described herein.

FIG. 3 is a representation of an algorithm for optimising feature weights.

FIG. 4a is a representation of a multidimensional standard curve.

FIG. 4b is a representation of a resulting quantification curve obtained after dimensionality reduction through principal component regression.

FIG. 5 shows a mean of outliers in the feature space, and an orthogonal projection of the mean of the outliers onto the standard curve.

FIG. 6a is a representation of a view of the feature space along an axis of the multidimensional standard curve, by projecting onto a plane that is perpendicular to the standard curve.

FIG. 6b is a representation of the resulting projected points according to FIG. 6a.

FIG. 6c is a representation of a transformation of the orthogonal view of the feature space of FIG. 6b into a new space where the Euclidean distance is equivalent to the Mahalanobis distance in the original space.

FIG. 7 shows a histogram of Mahalanobis distance squared, for an entire training set superimposed with a χ²-distribution with 2 degrees of freedom.

FIG. 8a shows a multidimensional pattern associated with temperature.

FIG. 8b shows a multidimensional pattern associated with primer mix concentration.

FIG. 8c shows a variation of training data points along the axis of the multidimensional standard curve, for low concentrations of nucleic acids.

FIG. 9 is an illustration of experimental workflow and comparison of real-time uni-dimensional vs multi-dimensional standard curves.

FIG. 10 shows multidimensional standard curves constructed using a single primer mix (by multiplex real-time PCR) fix for four target genes using Cₜ, C_{y} and −log₁₀(F₀).

FIG. 11 shows real-time amplification data and melting curve analysis (for validation purposes) for the training samples.

FIG. 12 shows a Mahalanobis space for each of four multidimensional standard curves.

FIG. 13 is a representation of an example networked computer system in which embodiments of the disclosure can be implemented.

FIG. 14 is a representation of an example computing device such as the ones shown in FIG. 13.

FIGS. 15a-15d show melting curves analysis for the training data (15a), outliers (15b), primer concentration experiment (15c) and temperature variation experiment (15d), according to an example.

FIG. 16 shows average Mahalanobis distance from standard points to sample tests in an example. Which is used to classify the samples into blaOXA-48, blaNDM, blaVIM and blaKPC genes, based only on real-time amplification curves obtained by the multiplex PCR assay.

### DETAILED DESCRIPTION

The structure of the disclosure is as follows. In order to understand the proposed framework, it is useful to have an overall picture of what is done in the conventional approach in the same language. First, the conventional approach and then the proposed multidimensional framework are presented. For easier comprehension, the theory and benefits of the disclosed method are explained and discussed. Further, by way of example, an example instance of this new method is given, with a set of real-time data using lambda DNA as a template, and specific applications of the disclosed methods are explored.

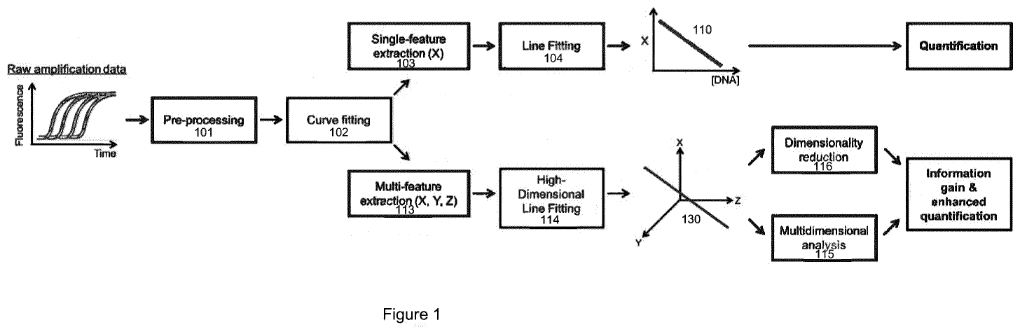

FIG. 1 is a block diagram showing the disclosed multi-dimensional method (bottom branch) compared to a conventional method (top branch) for absolute quantification of target based on serial dilution of a known target.

Conventional Approach

In a conventional method, raw amplification data for several known concentrations of the target is typically pre-processed and fitted with an appropriate curve. A single feature such as the cycle threshold, Cₜ, is extracted from each curve. A line is fitted to the feature vs concentration such that unknown sample concentrations can be extrapolated. Here, two terms, namely training and testing (as used in the field of Machine Learning), are used to describe the construction of a standard curve 110 and quantifying unknown samples respectively. Within the conventional approach for quantification, training using a first set of data relating to melting temperatures of samples having known characteristics is achieved through 4 stages: pre-processing 101, curve fitting 102, single linear feature extraction 103 and line fitting 104, as illustrated in the upper branch of FIG. 1.

Pre-processing 101 can be optionally performed to reduce factors such as background noise such that a more accurate comparison amongst samples can be achieved.

Curve fitting 102 (e.g. using a 5-parameter sigmoid, an exponential model, and/or linear interpolation) is optional, and beneficial given that amplification curves are discrete in time/temperature and most techniques require fluorescence readings that are not explicitly measured at a given time/temperature instance.

Feature extraction 103 involves selecting and determining a feature (or “characteristic”, e.g. Cₜ, C_{y}, −log₁₀(F₀), FDM, SDM) of the target data.

Line (or curve) fitting 104 involves fitting a line (or curve) 110 to the determined feature data versus target concentration.

Examples of pre-processing 101 include baseline subtraction and normalization. Examples of curve fitting 102 include using a 5-parameter sigmoid, an exponential model, and linear interpolation. Examples of features extracted in the feature extraction 103 step include Cₜ, C_{y} or −log₁₀(F₀). Examples of line fitting 104 techniques include principal component analysis, and random sample consensus (RANSAC).

Testing of unknown samples (i.e. quantifying target concentration in unknown samples, based on second data relating to the melting temperature of a target comprised in the unknown sample) is accomplished by using the same first 3 blocks (pre-processing 101, curve fitting 102, linear feature extraction 103) as training, and using the line 110 generated from the final line fitting 104 step during training in order to quantify the samples.

Proposed Method

The proposed method builds on the conventional techniques described in the above paragraph, by increasing the dimensionality of the standard curve (against which data is compared in the testing phase) in order to explore, research and take advantage of using multiple features together. This new framework is presented in the lower branch of FIG. 1.

For training, in this example embodiment there are 6 stages: pre-processing 101, curve fitting 102, multi-feature extraction 113, high dimensional line fitting 114, multidimensional analysis 115, and dimensionality reduction 116. Testing follows a similar process: pre-processing 101, curve fitting 102, multi-feature extraction 113, multidimensional analysis 115, and dimensionality reduction 116. As for the conventional approach, pre-processing 101 and curve fitting 102 are optional, and with suitable multidimensional analysis techniques an explicit step of dimensionality reduction may also be rendered optional.

Again, examples of pre-processing 101 include baseline subtraction and normalization, and examples of curve fitting 102 include using a 5-parameter sigmoid, an exponential model, and linear interpolation. Examples of features extracted in the multi-feature extraction 113 step include Cₜ, C_{y}, −log₁₀(F₀), FDM, SDM. Examples of high-dimensional line fitting 114 techniques include principal component analysis, and random sample consensus (RANSAC). Examples of multidimensional analysis 115 techniques include calculating a Euclidean distance, calculating confidence bounds, weighting features using scalars αᵢ, as further described below. Examples of dimensionality reduction 116 techniques include principal component regression, calculating partial least-squares, and projecting onto original features, as further described below.

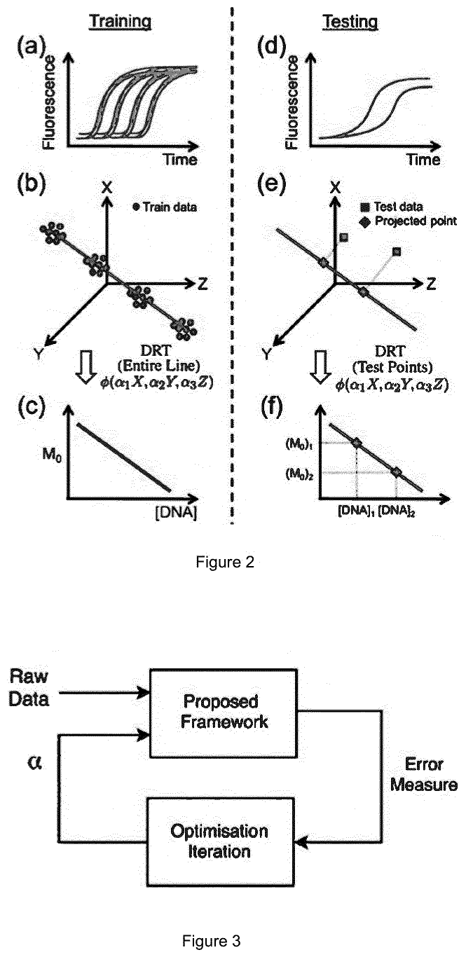

FIGS. 2a-2c illustrate the process of training and FIGS. 2d-2f show testing using the multidimensional approach. Starting with training, FIG. 2a shows processed and curve-fitted real-time nucleic acid amplification curves obtained from a conventional qPCR instrument by serially diluting a known nucleic acid target to known concentrations. In contrast with the conventional training, instead of extracting a single linear feature, multiple features denoted using the dummy labels X, Y and Z are extracted from the processed amplification curves. Therefore, each amplification curve has been reduced to a number of sets of 3 values (e.g. X₁, Y₁ and Z₁) and, consequently, can be viewed as a number of points plotted against each other in 3-dimensional space as shown in FIG. 2b. It is important to stress that although this is a 3-D example (in order to visualize the process), optionally any number of features can be chosen. Given that all the features in this example have been chosen such that they are linearly related to initial concentration, the training data forms a 1-D line in 3-D space, and this line is then approximated using high-dimensional line fitting 114 to generate what is termed the multidimensional standard curve 130. Although, the data forms a line, it is important to understand that data points do not necessarily lie exactly on the line. Consequently, there is considerable room for exploring this multidimensional space, referred to as the feature space, which will be discussed herein. Although in this example, only linear features (i.e. features linearly related to target concentration) are considered, the disclosed method can be applied to non-linear features by making appropriate changes. For quantification purposes, the multidimensional standard curve is mapped into a single dimension, M₀, which function is linearly related to the initial concentration of the target. In order to distinguish the curve described by such a function from conventional standard curves, it is referred to here as the quantification curve 150. This is achieved using dimensionality reduction techniques (DRT) as illustrated in FIG. 2c. Mathematically, this means that DRTs are multivariate functions of the form: M₀=φ(X,Y,Z) where φ(·):R3→R. In fact, given that scaling features does not affect linearity, M₀ can be mathematically expressed as M₀=φ(α1X,α2Y,α3Z) where i∈{1,2,3}, are scalar constants.

Once training is complete, at least one further (e.g. unknown) sample can then be analyzed (e.g. quantified and/or classified) through testing as follows. Similar to training, processed amplification data (FIG. 2d) and their respective corresponding point in the feature space (FIG. 2e) is shown. Given that test points may lie anywhere in the feature space, it is necessary to project them onto the multidimensional standard curve 130 generated in training. Using the DRT function, φ, which was produced in training, M₀ values for each test sample can be obtained. Subsequently, absolute quantification is achieved by extrapolating the initial concentration based on the quantification curve 150 in FIG. 2f. It will be noted that data relating to these further samples can be used to refine the multidimensional standard curve 130 (e.g. by re-fitting a line to a plurality of points defined in N-dimensional space by the extracted features, including both the original set of training data, and the data relating to the further sample).

Given that this higher dimensional space has not previously been disclosed, it is effective to highlight the degrees of freedom within this new framework that were non-existent when observing the quantification process through the conventional lens. The following advantages arise:

Advantage 1. The weight of each extracted feature can be controlled by the scalars, α1, . . . αn. There are two main observations of this degree of freedom. The first observation is that features that have poor quantification performance can be suppressed by setting the associated a to a small value. This introduces a very useful property of the framework which is referred to as the separation principle. The separation principle means that including features to enhance multidimensional analyses does not have a negative impact on quantification performance if the a's are chosen appropriately. Optimization algorithms can be used to set the a's based on an objective function. Therefore, the performance of the quantification using the proposed framework is lower bounded by the performance of the best single feature for a given objective. The second observation is that no upper bound exists on the performance of using several scaled features. Thus, there is a potential to outperform single features as shown in this report.

Advantage 2. The versatility of this multidimensional way of thinking means that there are multiple methods for dimensionality reduction such as: principal component regression, partial-least squares regression, and even projecting onto a single feature (e.g. using the standard curve 110 used in conventional methods). Given that DRTs can be nonlinear and take advantage of multiple features, predictive performance may be improved.

Advantage 3. Training and testing data points do not necessarily lie perfectly on a straight line as they did in the conventional technique. This property is the backbone behind why there is more information in higher dimensions. For example, the closer two points are in the feature space, the more likely that their amplification curves are similar (resembling a Reproducing Kernel Hilbert Spaces). Therefore, a distance measure in the feature space can provide a means of computing a similarity measure between amplification curves. It is important to understand that the distance measure is not necessarily, and in reality unlikely to be, linearly related to the similarity measure. For example, it is not necessarily true that a point twice as far from the multidimensional standard curve is twice as unlikely to occur. This relationship can be approximated using the training data itself. In the case of training, a similarity measure is useful to identify and remove outliers that may skew quantification performance. As for testing, the similarity measure can give a probability that the unknown data is an outlier of the standard curve, i.e. non-specific or due to a qPCR artefact, without the need of post-PCR analyses such as melting curves or agarose gels.

Advantage 4. The effect of changes in reaction conditions, such as annealing temperature or primer mix concentration, can be captured by patterns in the feature space. Uncovering these trends and patterns can be very insightful in understanding the data. This is also possible in the conventional case, e.g. how Cₜ varies with temperature, however since reaction conditions affect different features differently, in the proposed multidimensional technique conclusions can be drawn with higher confidence e.g. if a pattern is observed in multidimensional space. For example, consider the following: a change in temperature, ΔT, causes a different change for different features, e.g. ΔX, ΔY and ΔZ. Therefore, if (as in the conventional technique) only a single feature, X, is used and a variation ΔX is observed then it is unlikely to capture the source of the variation, i.e. AT, with high confidence. Whereas, considering multiple features (as in the proposed multidimensional technique) and observing ΔX, ΔY and ΔZ simultaneously, can provide more confidence that the source is due to ΔT.

An extension of advantage 4 is related to the effect of variations in target concentration. Clearly, the pattern for varying target concentration is known: along the axis of the multidimensional standard curve 130. Therefore, the data itself is sufficient to suggest if a particular sample is at a different concentration than another. This is significant, since it allows variations amongst replicates (which are possible due to experimental errors such as dilution and mixing) to be identified and potentially compensated for. This is of particular importance for low concentrations wherein such errors are typically more significant. It is interesting to observe that if multiple features are used, and the DRT is chosen such that the multidimensional curve is projected onto a single feature, e.g. Cₜ, then the quantification performance is similar as for the conventional process (e.g. a special instance of the proposed framework, wherein only a single feature is used) yet the opportunities and insights obtained as a result of employing a multidimensional space still remain.

Example Method

It has been established that each step in the proposed method, as seen in the lower branch of FIG. 1, can be implemented using several different techniques, given as examples in the Figure. The specific techniques used for each block can be application dependent, however specific example methods are described herein to illustrate the power and versatility of this method. It will nevertheless be understood that the described method is not limited to those specific examples.

Pre-Processing 101

The only pre-processing 101 performed in this example is background subtraction. This is accomplished using baseline subtraction: removing the mean of the first 5 fluorescence readings from every amplification curve. In other embodiments, however, pre-processing can be omitted, or other or additional pre-processing steps such as normalization can be carried out, and more advanced pre-processing steps can optionally be carried out so improve performance and/or accuracy.

Curve Fitting 102

An example model for curve fitting is the 5-parameter sigmoid (Richards Curve) given by:

$$F ( x ) = {F}_{b} + \frac{{F}_{max}}{{( 1 + {e}^{- ( x - c ) / b} )}^{d}} \qquad ( 1 )$$

Where x is the cycle number, F(x) is the fluorescence at cycle x, F_{b} is the background fluorescence, Fₘₐₓ is the maximum fluorescence, c is the fractional cycle of the inflection point, b is related to the slope of the curve, and d allows for an asymmetric shape (Richard's coefficient).

An example optimization algorithm used to fit the curve to the data is the trust-region method and is based on the interior reflective Newton method. Here, the trust-region method is chosen over the Levenberg-Marquardt algorithm since bounds for the 5 parameters can be chosen in order to encourage a unique and realistic solution. Example lower and upper bounds for the 5 parameters, [F_{b}, Fₘₐₓ, c, b, d], are given as: [−0.5, −0.5, 0, 0, 0.7] and [0.5, 0.5, 50, 100, 10] respectively.

Multi Feature Extraction 113

The number of features, n, that can be extracted is arbitrary, however 3 features have been chosen in this example in order to enhance visualization of each step of the framework: Cₜ, C_{y} and −log₁₀(F₀), for ease of explanation. As a result, in this example, each point in the feature space is a vector in 3-dimensional space,

e.g. p=[Cₜ,C_{y},−log₁₀(F₀)]^{T}

where [·]^{T} denotes the transpose operator.

Note that by convention, vectors are columns and are bold lowercase letters. Matrices are bold uppercase. The details of these features are not the focus of this disclosure, and so will not be described further herein, it being assumed that the reader is familiar with said details.

High-Dimensional Line Fitting 114

When constructing a multidimensional standard curve, a line must be fitted in n-dimensional space. This can be achieved in multiple ways such as using the first principal component in principal component analysis (PCA) or techniques robust to outliers such as random sample consensus (RANSAC) if there is sufficient data. This example uses the former (PCA) since a relatively small number of training points are used to construct the standard curve.

Distance and Similarity Measure (Multi-Dimensional Analysis 115)

There are two distance measures given as examples in this disclosure: Euclidean and Mahalanobis distance, although it will be appreciated that other distance measures can be used.

The Euclidean distance between a point, p, and the multidimensional standard curve can be calculated by orthogonally projecting a point onto the multidimensional standard curve 130 and then using simple geometry to calculate the Euclidean distance, e:

$$\begin{matrix}P = Φ ( p , q 1 , q 2 ) = \frac{{( p - q 1 )}^{T} ( q 2 - q 1 )}{{( q 2 - q 1 )}^{T} ( q 2 - q 1 )} & ( 2 ) \\ e = \vert ( p - q 1 ) - ( q 1 + P \cdot ( q 2 - q 1 ) ) \vert & ( 3 )\end{matrix}$$

where Φ computes the projection of the point p∈Rⁿ onto the multidimensional standard curve, the points q1,q2∈Rⁿ are any two distinct points that lie on the standard curve, and |·| denotes the absolute value operator.

The Mahalanobis distance is defined as the distance between a point, p, and a distribution, D, in multidimensional space. Similar to the Euclidean distance, a point is first projected onto the multidimensional standard curve 130 and the following formula is applied to compute the Mahalanobis distance, d:

d={square root over ((p−P·(q₂−q₁)^{T}Σ⁻¹(p−P·(q₂−q₁))} (4)

where p, P, q1 and q2 are given in equation (2), and Σ is the co-variance matrix of the training data used to approximate the distribution D.

In order to convert the distance measure into a similarity measure, it can be shown that if the data is approximately normally distributed then the Mahalanobis distance squared, i.e. d², follows an χ²-distribution. Therefore, an χ²-distribution table can be used to translate a specific p-value into a distance threshold. For instance, for a χ²-distribution with 2 degrees of freedom, a p-value of 0.05 and 0.01 correspond to a squared Mahalanobis distance of 5.991 and 9.210 respectively.

Feature weights.

As mentioned previously, different weights, a, can be assigned to each feature. In order to accomplish this, a simple optimization algorithm can be implemented. Equivalently, an error measure can be minimized. FIG. 3 is an illustration of how an optimization algorithm can be used to find optimal parameters, a, for the disclosed method. In this example, the error measure to minimize is the figure of merit described in the following subsection. By way of example, a suitable optimization algorithm is the Nelder-Mead simplex algorithm with weights initialized to unity, i.e. beginning with no assumption on how good features are for quantification. This is a basic algorithm and only 20 iterations are used to find the weights so that there is little computational overhead.

Dimensionality Reduction 116

In this example, principal component regression is used, e.g. M₀=P from equation (2), and it is compared with projecting the standard curve onto all three dimensions, i.e. Cₜ, C_{y} and −log₁₀(F₀).

Evaluating Standard Curves

In consistency with the existing literature on evaluating standard curves, relative error (RE) and average coefficient of variation (CV) can, by way of example, be used to measure accuracy and precision respectively. The CV for each concentration can be calculated after normalizing the standard curves such that a fair comparison across standard curves is achieved. The formula for the two measures are given by:

$$RE = \frac{1}{n} {\sum }_{i = 1}^{n} ( 100 \times ( \frac{{\hat{x}}_{i}}{{x}_{i}} - 1 ) ) \qquad ( 5 )$$

where n is the number of training points, i is the index of a given training point, xi is the true concentration of the i^{th} training data, x\hat{ }ᵢ is the estimate of xi using the standard curve.

$$CV = \frac{1}{m} {\sum }_{j = 1}^{m} ( 100 \times \frac{std ( {\hat{x}}^{j} )}{mean ( {\hat{x}}^{j} )} ) \qquad ( 6 )$$

where m is the number of concentrations, j is the index of a given concentration and x is a vector of estimated concentrations for a given concentration indexed by j. The functions std(·) and mean(·) perform the standard deviation and mean of their vector arguments respectively.

Referring to the field of Statistics, this example also uses the “leave one-out cross validation” (LOOCV) error as a measure for stability and overall predictive performance. Stability refers to the predictive performance when training points are removed. The equation for calculating the LOOCV is given as:

$$LOOCV = \sqrt{\frac{1}{n} {\sum }_{i = 1}^{n} {( {z}_{i} - {\hat{z}}_{i} )}^{2}} \qquad ( 7 )$$

where n is the number of training points, i is the index of a given training point, zᵢ is a vector of the true concentration for all training points except the i^{th} training point and z\hat{ }ᵢ is the estimate of zᵢ generated by the standard curve without the i^{th} training point.

In order for the optimization algorithm for computing a to simultaneously minimize the three aforementioned measures, it is convenient to introduce a figure of merit, Q, to capture all of the desired properties. Therefore, Q is defined as the product between all three errors and can be used to heuristically compare the performance across quantification methods.

Q=RE×CV×LOOCV (8)

### Example Fluorescence Datasets

Several DNA targets were used for qPCR amplification by way of example:

(i) Synthetic double-stranded DNA (gblocks Fragments Genes, Integrated DNA Technologies) containing phage lambda DNA sequence was used to construct and evaluate the standards curves (DNA concentration ranging from 10² to 10⁸ copies per reaction). See Appendix A.

(ii) Genomic DNA isolated from pure cultures of carbapenem-resistant (A) Klebsiella pneumoniae carrying bla_{OXA-48}, (B) Escherichia coli carrying bla_{NDM} and (C) Klebsiella pneumoniae carrying bla_{KPC} were used for the outlier detection experiments. See Appendix B.

(iii) Phage lambda DNA (New England Biolabs, Catalog #N3011S) was used for primer variation experiment (final primer concentration ranging from 25 nM/each to 850 nM/each) and temperature variation experiments (annealing temperature ranging from 52° C. to 72° C.

All oligonucleotides used in this example were synthesised by IDT (Integrated DNA Technologies, Germany) and are shown in Table 1. The specific PCR primers for lambda phage were designed in-house using Primer3 (http://biotools.umassmed.edu/bioapps/primer3_www.cgi), whereas the primer pairs used for the specific detection of carbapenem resistance genes were taken from Monteiro et al 2012. Real-time PCR amplifications were conducted using FastStart Essential DNA Green Master (Roche) according to the manufacturer's instructions, with variable primer concentration and a variable amount of DNA in a 54 final reaction volume. Thermocycling was performed using a LightCycler 96 (Roche) initiated by a 10 min incubation at 95° C., followed by 40 cycles: 95° C. for 20 sec; 62° C. (for lambda) or 68° C. (for carbapenem resistance genes) for 45 sec; and 72° C. for 30 sec, with a single fluorescent reading taken at the end of each cycle. Each reaction combination, starting DNA and specific PCR amplification mix, was conducted in octuplicate. All the runs were completed with a melting curve analysis to confirm the specificity of amplification and lack of primer dimer. The concentrations of all DNA solutions were determined using a Qubit 3.0 fluorometer (Life Technologies). Appropriate negative controls were included in each experiment.

TABLE 1
Specific PCR primers used in this example

| | | | Amplicon |
|---|---|---|---|
| | Primer | | size |
| Target | name | Sequence (5-3) | (hp) |
| lambda | lambda-F | CGGTGGCAAGGGTAATGAGG | 72 |
| | lambda-R | TCAGCATCCCTTTCGGCATA | |
| bla_{OXA-48} | OXA-48-F | TGTTTTTGGTGGCATCGAT | 177 |
| | OXA-48-R | GTAAMRATGCTTGGTTCGC | |
| bla_{NDM} | NDM-F | TTGGCCTTGCTGTCCTTG | 82 |
| | NDM-R | ACACCAGTGACAATATCACCG | |
| bla_{KPC} | KPC-F | TTACTGCCCGTTGACGCCCAATCC | 785 |
| | KPC-R | TTACTGCCCGTTGACGCCCAATCC | |

Results

The following example results illustrate the aforementioned advantages of the proposed framework using an example instance of the method as described above. Given that there is a separation principle between quantification performance and insights in the feature space, this section is split into two parts: quantification performance and multidimensional analysis. The first part shows the results that arose from the two degrees of freedom introduced in advantage 1 & 2 and the latter explores advantage 3 & 4 regarding interesting observations in multidimensional space.

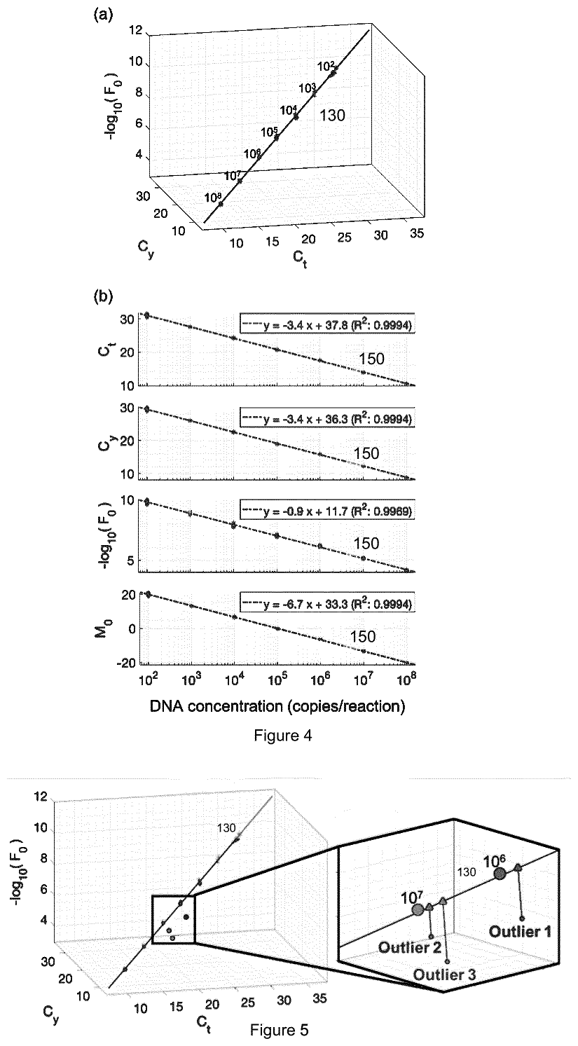

FIG. 4 shows the multidimensional standard curve 130 and quantification using information from all features. In FIG. 4a, a multidimensional standard curve 130 is constructed using Ct, Cy and −log 10(F0) for lambda DNA with concentration values ranging from 10² to 10⁸ (top right to bottom left). Each concentration was repeated 8 times. The line fitting was achieved using principal component analysis. In FIG. 4b, the quantification curves 150 were obtained by dimensionality reduction of the multidimensional standard curve using principal component regression.

Quantification Performance

In this example, synthetic double-stranded DNA was used to construct a multidimensional standard curve 130 and evaluate its quantification performance relative to single feature methods. The resulting multidimensional standard curve 130, constructed using the features Cₜ, C_{y} and −log₁₀(F₀), is visualized in FIG. 4a. The computed features and curve fitting parameters for each amplification curve grouped by concentration, ranging from 10² to 10⁸, is presented in Appendix C. FIG. 4b shows the resulting uni-dimensional quantification curve 150 obtained after dimensionality reduction 116 through principal component regression. For comparison, the standard curves for the conventional examples are computed by projecting the multidimensional standard curve onto each feature, as listed in Appendix D.

In this example, the optimal feature weights, a, to control the contribution of each feature to quantification, after 20 iterations of the optimization algorithm, converged to α=[1.6807,1.0474,0.0134] where the weights correspond to Cₜ, C_{y} and −log₁₀(F₀) respectively. This result is readily interpretable and it suggests that −log₁₀(F₀) exhibits the poorest quantification performance amongst the three features; as consistent with the existing knowledge. It is important to stress again that although the weight of −log₁₀(F₀) is suppressed relative to the other features to improve quantification, there is still a lot of value in keeping it as it can uncover trends in multidimensional space: as will become apparent later.

The performance measures and figure of merit, Q, for this particular instance of the proposed framework against the conventional instance is given in Table 2. A breakdown of each calculated error grouped by concentration is provided in Appendix D. It can be observed that Cₜ offers the smallest RE, i.e. accuracy, whereas M₀ outperforms the other methods in CV and LOOCV, i.e. precision and overall prediction. In terms of the figure of merit, combining all of the errors, this arbitrary realisation of the framework enhanced quantification by 6.8%, 25.6% and 99.3% compared to Cₜ, C_{y} and −log₁₀(F₀) respectively.

TABLE 2
Performance measures for quantification methods used in
this example along with a heuristic figure of merit, Q.

| | RE (%) | CV (%) | LOOCV (%) | Fig. of Merit, Q |
|---|---|---|---|---|
| Cₜ | 7.70 ± 5.87 | 0.97 ± 0.77 | 9.52 ± 8.20 | 71.1 ± 37.22 |
| C_{y} | 8.01 ± 6.5 | 1.11 ± 1.28 | 9.47 ± 8.61 | 84.6 ± 71.46 |
| F₀ | 21.86 ± 7.50 | 7.76 ± 12.78 | 26.3 ± 9.39 | 4460 ± 903.08 |
| M₀ | 7.76 ± 6.06 | 0.90 ± 0.74 | 9.42 ± 8.34 | 65.8 ± 37.37 |

RE = relative error, CV = coefficient of variation, LOOCV = leave-one-out cross validation.

Multidimensional Analysis

Given that the feature space is a new concept, there is room to explore what can be achieved. In this section the concept of distance in the feature space is explored and is demonstrated through an example of outlier detection. Furthermore, it is shown that in this example a pattern exists in the feature space when altering reaction conditions.

FIG. 5 shows outliers in the feature space, specifically the multidimensional standard curve 130 for lambda DNA along with three carbapenemase outliers: blaOXA, blaNDM and blaKPC. On the right of FIG. 5 is shown a zoomed view into the region of the feature space with the mean of the replicates and the projection of the outliers onto the standard curve.

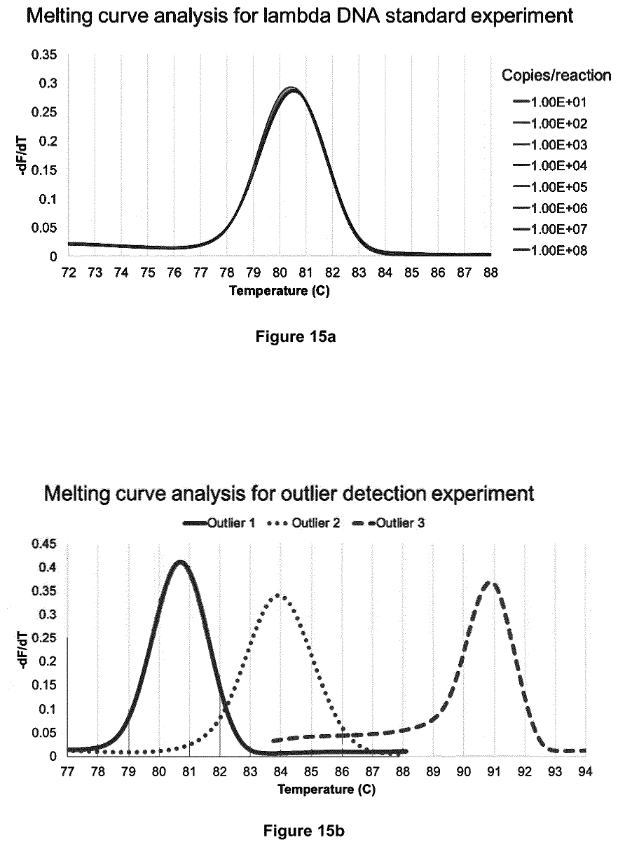

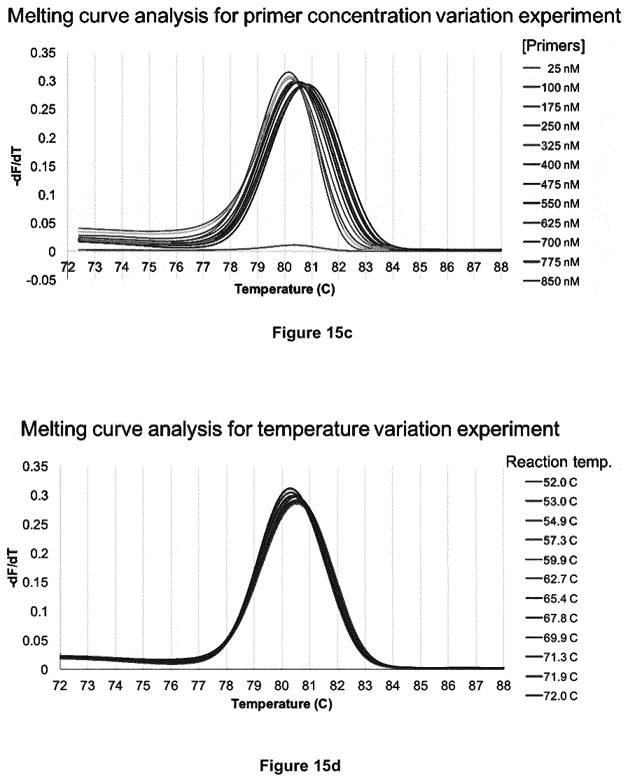

In this example, genomic DNA carrying carbapenemase genes, namely bla_{OXA}, bla_{NDM} and bla_{KPC}, are used as deliberate outliers for the multidimensional standard curve 130. FIG. 5 shows the mean of the outliers in the feature space. The computed features and curve-fitting parameters for outlier amplification curves in this example are shown in Appendix E, and specificity of the outliers is confirmed using a melting curve analysis as presented in Appendix F and FIGS. 15a-15d. Given that the outlier test points do not lie exactly on the multidimensional standard curve 130, FIG. 5 also shows the orthogonal projection of the mean of the outliers onto the multidimensional standard curve 130; as described in the proposed framework.

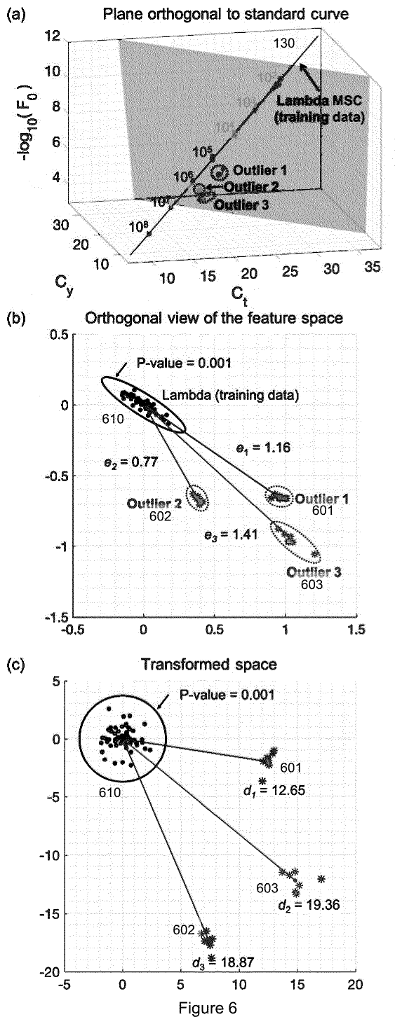

In order to fully capture the position of the outliers in the feature space, it is convenient to view the feature space along the axis of the multidimensional standard curve 130. This is possible by projecting data points in the feature space onto the plane perpendicular to the multidimensional standard curve 130 as illustrated in FIG. 6a. The resulting projected points are shown in FIG. 6b.

FIG. 6 shows a multidimensional analysis using the feature space for clustering and detecting outliers. In particular, FIG. 6a shows a multidimensional standard curve 130 using Cₜ, C_{y} and −log₁₀(F₀) for lambda DNA with concentration values ranging from 10² to 10⁸ (top right to bottom left). An arbitrary hyperplane orthogonal to the standard curve is shown in grey. FIG. 6b shows a view of the feature space when all the data points have been projected onto the aforementioned hyperplane. The data points consist of training standard points and outliers corresponding to blaOXA, blaNDM and blaKPC. Errors corresponding to the Euclidean distance, e, from the multidimensional standard curve to the mean of the outliers is given by eOXA=1.16, eNDM=0.77 and eKP C=1.41. The 99.9% confidence corresponding to a p-value of 0.001 is shown with a solid black line. FIG. 6c shows a transformed space where the Euclidean distance, d, is equivalent to the Mahalanobis distance in the orthogonal view. The black circle corresponds to a p-value of 0.001.

It can be observed that all three outliers 601, 602, 603 can be clustered and clearly distinguished from the training data 610. Furthermore, in this example, the Euclidean distance, e, from the multidimensional standard curve 130 to the mean of the outliers is given by e_{OXA}=1.16, e_{NDM}=0.77 and e_{KPC}=1.41. Given that in this example the furthest training point from the multidimensional standard curve 130 in terms of Euclidean distance is 0.22: the ratio between e_{OXA}, e_{NDM}, e_{KPC} and 0.22 is given by 5.27, 3.5, 6.41 respectively. Therefore, this ratio can be used as a similarity measure and the three clusters could be classified as outliers. However, this similarity measure has two implicit assumptions: (i) The data follows a uniform probability distribution. That is, a point twice as far is twice as likely to be an outlier. This assumption is typically made when there is not enough information to infer a distribution. (ii) Distances in different directions (e.g. along difference axes) are equally likely. This is intuitively untrue in the feature space because a change along one direction, e.g. Cₜ, does not impact the amplification curve as much as a change in another direction, e.g. −log₁₀(F₀). It is important to emphasise that directions in the feature space contain information regarding how much amplification kinetics change and therefore direct comparisons between amplification reactions should be made along the same direction. This information is not captured in the aforementioned previous (unidimensional) data analysis.

In order to tackle the two aforementioned assumptions, the Mahalanobis distance, d, can be used. Clearly, by observing FIG. 6b, the data predominantly varies in a given direction. The Mahalanobis distance can be computed directly using equation (4). In order to visualize the Mahalanobis distance, the orthogonal view of the feature space (FIG. 6b) can be transformed into a new space (“Transformed space” in FIG. 6c) wherein the Euclidean distance, e, is equivalent to the Mahalanobis distance, d, in the original space (i.e. the space illustrated in FIG. 6b). It can be seen from FIG. 6c that data in all directions are equiprobable, i.e. the training data 610 forms a circular distribution. The Mahalanobis distance, d, from the multidimensional standard curve 130 to the mean of the outliers 601, 602, 603 is given by d_{OXA}=12.65, d_{NDM}=18.87 and d_{KPC}=19.36. In comparison to the Euclidean distances, it is observed that when considering the distribution of the data, the position of the outliers significantly change. As an example, based on Euclidean distance, blaNDM 601 is the closest outlier whereas using the Mahalanobis distance suggests bla_{OXA} 603.

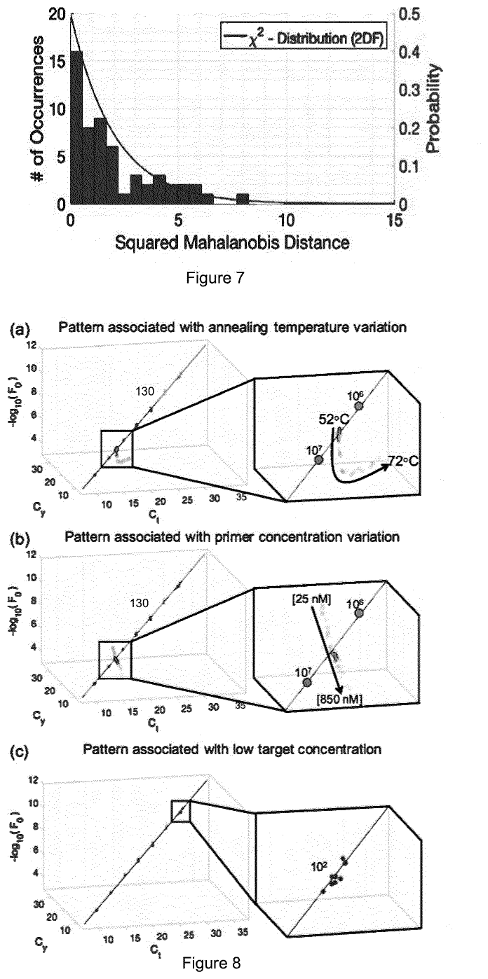

A useful property of the Mahalanobis distance is that its squared value follows a χ²-distribution if the data is approximately normally distributed. Therefore, the distance can be converted into a probability in order to capture the non-uniform distribution. FIG. 7 shows a histogram of Mahalanobis distance, d, squared, for the entire training set, superimposed with a χ²-distribution with 2 degrees of freedom. In this example, based on the χ²-distribution table, any point further than about 3.717 is 99.9% (p-value<0.01) likely to be an outlier. FIG. 7 thus shows the data distribution, in terms of a histogram of the Mahalanobis distance squared of all training data points used in constructing the multidimensional standard curve superimposed with a x2-distribution with 2 degrees of freedom. Since all the outliers have a Mahalanobis distance significantly greater than about 3.717, they can be detected as outliers. Other distances (greater or smaller) can be chosen as a criterion for testing against the Mahalanobis distance, depending on the level of confidence required as to whether points are inliers or outliers. A distance of 3.717 has been illustrated since that corresponds to a probability of 99%, but distances corresponding to other probabilities such as 80%, 95%, 99.9% can also be chosen.

A second example multidimensional analysis (as shown in FIG. 8) is concerned with observing patterns with respect to reaction conditions. FIG. 8 shows patterns associated with changing reaction conditions. The multidimensional standard curve in all plots are using Cₜ, C_{y} and −log₁₀(F₀) for lambda DNA with concentration values ranging from 10² to 10⁸ copies/reaction (top right to bottom left). In FIG. 8a, the magnified image shows the effect of changing the reaction temperature from 52° C. to 72° C. for lambda DNA at 5×10⁶ copies/reaction. In FIG. 8b, the magnified image shows the effect of changing the primer mix concentration from 25 nM to 850 nM for each primer for lambda DNA at 5×10⁶ copies/reaction. In FIG. 8c, the magnified image shows the individual training sample location in the feature space for a given low concentration: 10² copies/reaction

In the illustrated example, annealing temperature and primer mix concentration have been chosen to illustrate the idea. Specificity of the qPCR is not affected, as shown with melting curve analyses (see Appendix F and FIGS. 15a-15d). FIG. 8a shows the effect of annealing temperature on the standard curve. Temperatures ranging from 52.0° C. to 69.9° C. only affect −log₁₀(F₀) whereas changes from 69.9° C. to 72.0° C. affect mostly Cₜ and C_{y} (see Appendix G). Similarly, FIG. 8b shows there is a pattern associated with primer mix concentration: the variation from 25 to 850 nM for each primer is observed predominantly along the −log₁₀(F₀) direction (see Appendix H). Both experiments show that Cₜ and C_{y} are more robust to changes in annealing temperature and primer mix concentration, which is good for quantification performance. Furthermore, the patterns are observed in the feature space predominantly due to −log₁₀(F₀).

Based on this finding, the previous (unidimensional) way of proceeding would indicate the use of Cₜ or C_{y} for subsequent experiments. However, it has been realised that this implies a loss of information contained in patterns generated by −log₁₀(F₀). Therefore, the proposed multidimensional approach combines features that are beneficial for quantification performance and pattern recognition: preserving all information without compromising quantification performance.

Finally, a further interesting observation is that for low concentrations of nucleic acids, there is a variation of training data points along the axis of the multidimensional standard curve 130 as seen in FIG. 8c. Thus, it can be hypothesized that the variation is due to fluctuations in concentration as opposed to changes in reaction kinetics. There are two implications of this assumption: (i) all the points are inliers and thus likely to be specific without the need of resource consuming post-PCR analyses. Specificity is confirmed using a melting curve analysis, as for example given in Appendix F; (ii) The outcome of absolute quantification is based on 3 features as opposed to a single feature which implies an increased confidence in the estimated target concentration.

Although the disclosed framework has been described as considering features that are linearly related to initial target concentration, that example design choice was chosen so as to reduce the complexity of the analysis, however other features such as non-linearly related features can optionally be used.

Additionally, it will be noted that if two unrelated PCR reactions exhibit a perfectly symmetric sigmoidal amplification curve, their respective standard curves may potentially overlap, and thus a question arises as to whether sufficient information might be captured between amplification curves in order to distinguish them in the feature space. However, such an effect can be mitigated from a molecular perspective by tuning the chemistry in order to sufficiently change amplification curves without compromising the performance of the reaction (e.g. speed, sensitivity, specificity etc).

### CONCLUSION

In conclusion, this disclosure presents a versatile method, multidimensional standard curve and feature space, which enable techniques and advantages that were not previously realisable. It has been illustrated that an advantage of using multiple features is improved reliability of quantification. Furthermore, instead of trusting a single feature, e.g. Cₜ, other features such as C_{y} and −log₁₀(F₀) can be used to check if a quantification result is similar. The previous unidimensional way of thinking failed to consider multiple degrees of freedom and the resulting advantages that the versatile framework disclosed herein enables. There are thus four main capabilities that are enabled by the disclosed method:

(i) the ability to select multiple features and weight them based on quantification performance.

(ii) the flexibility of choosing an optimal mathematical method that maps multiple features into a single value representing target concentration. The first two capabilities lead to a separation principle which lower bounds the quantification performance of the framework to the best single feature, however the insights and multidimensional analyses from the multiple features still remain. It is interesting to observe that, for the example dataset used in this proposed approach, the gold standard Cₜ method outperformed the other single features. This is an example of why there is a technical prejudice against using other features, since the outcome is data dependent. The disclosed framework offers a method of absolute quantification without the need to select a specific feature with a guaranteed quantification performance. This disclosure shows that by using multiple features it is in fact possible to increase the quantification performance compared with the use of only single features.

(iii) enablement of applications such as outlier detection through the information gain captured by the elements of the feature space (e.g. distance measure, direction, distribution of data) that are typically meaningless or not considered in the previous unidimensional approach.

(iv) the ability to observe specific perturbations in reaction conditions as characteristic patterns in the feature space.

Example Application of the Disclosed Method

Absolute quantification of nucleic acids and multiplexing the detection of several targets in a single reaction both have, in their own right, significant and extensive use in biomedical related fields, especially in point-of-care applications. With previous approaches, the ability to detect several targets using qPCR scales linearly with the number of targets, and is thus an expensive and time-consuming feat. In the present disclosure, a method is presented based on multidimensional standard curves that extends the use of real-time PCR data obtained by common qPCR instruments. By applying the method disclosed herein, simultaneous single-channel multiplexing and robust quantification of multiple targets in a single well is achieved using only real-time amplification data (that is, using bacterial isolates from clinical samples in a single reaction without the need of post PCR operations such as fluorescent probes, agarose gels, melting curve analysis, or sequencing analysis). Given the importance and demand for tackling challenges in antimicrobial resistance, the proposed method is shown in this example to simultaneously quantify and multiplex four different carbapenemase genes: blaOXA-48, blaNDM, blaVIM and blaKPC, which account for 97% of the UK's reported carbapenemase-producing Enterobacteriaceae.

Quantitative detection of nucleic acids (DNA and RNA) is used for many applications in the biomedical field, including gene expression analysis, genetic disease predisposition, mutation detection and clinical diagnostics. One such application is in the screening of antibiotic resistance genes in bacteria: the emergence and spread of carbapenemase-producing enterobacteria (CPE) represents one of the most imminent threats to public health worldwide. Invasive infections with carbapenemase-resistant strains are associated with high mortality rates (up to 40-50%) and represent a major public health concern worldwide. Rapid and accurate screening for carriage of carbapenemase-producing Enterobacteriaceae (CPE) is essential for successful infection prevention and control strategies as well as bed management. However, routine laboratory detection of CPE based on carbapenem susceptibility is challenging: i) culture-based methods are convenient due to their ready availability and low cost, but their limited sensitivity and long turnaround time may not always be optimal for infection control practices; (ii) nucleic acid amplification techniques (NAATs), such as qPCR, provide fast results and added sensitivity and specificity compared with culture-based methods. However, these methodologies are often too expensive and require sophisticated equipment to be used as a screening tool in healthcare systems; and (iii) multiplexed NAATs have significant sensitivity, cost and turnaround time advantages, increasing the throughput and reliability of results, but the biotechnology industry has been struggling to meet the increasing demand for high-level multiplexing using available technologies. There is thus an unmet clinical need for new molecular tools that can be successfully adopted within existing healthcare settings.

Currently, qPCR is the gold standard for rapid detection of CPE and other bacterial infection. This technique is based on fluorescence-based data detection allowing kinetics of PCR amplification to be monitored in real-time. Different methodologies are used to analyze qPCR data, being the cycle-threshold (Cₜ) method the preferred approach for determining the absolute concentration of a specific target sequence. The Cₜ method assumes that the compared samples have similar PCR efficiency and it is defined as the number of cycles in the log-linear region of the amplification where there is significant detectable increase in fluorescence. Alternative methods have been developed to quantify template nucleic acids, including the standard curve methods, linear regression and non-linear regression models, but none of them allow simultaneous target discrimination. Multiplex analytical systems allow the detection of multiple nucleic acid targets in one assay and can provide the required speed for sample characterisation while still saving cost and resources. However, in a practical context, multiplex quantitative real-time PCR (qPCR) is limited by the number of detection channels of the real-time thermocycler and commonly rely on melting curve analysis, agarose gels or sequencing for target confirmation. These post-PCR processes increase diagnostic time, limit high throughput application and lead to amplicon contamination by laboratory environments. Therefore, there is an urgent need to develop simplified molecular tools which are sensitive, accurate and low-cost.

The disclosed method allows existing technologies to get as a return the benefits of multiplex PCR whilst reducing the complexity of CPE screening; resulting in cost reduction. This is due to the fact that the proposed method: (i) enables multi-parameter imaging with a single fluorescent channel; (ii) is compatible with unmodified oligonucleotides; and (iii) does not require post-PCR processing. This is enabled through the use of multidimensional standard curves, which in this example are constructed using Cₜ, C_{y} and −log₁₀(F₀) features extracted from amplification curves. In this example, we show that the described methodology can be successfully applied to CPE screening. This provides a proof-of-concept that several nucleic acid targets can be multiplexed in a single channel using only real-time amplification data. It will be appreciated nevertheless that the disclosed method can be applied to detection of any nucleic acid, and to detection of any pathogenic or non-pathogenic genomic material.

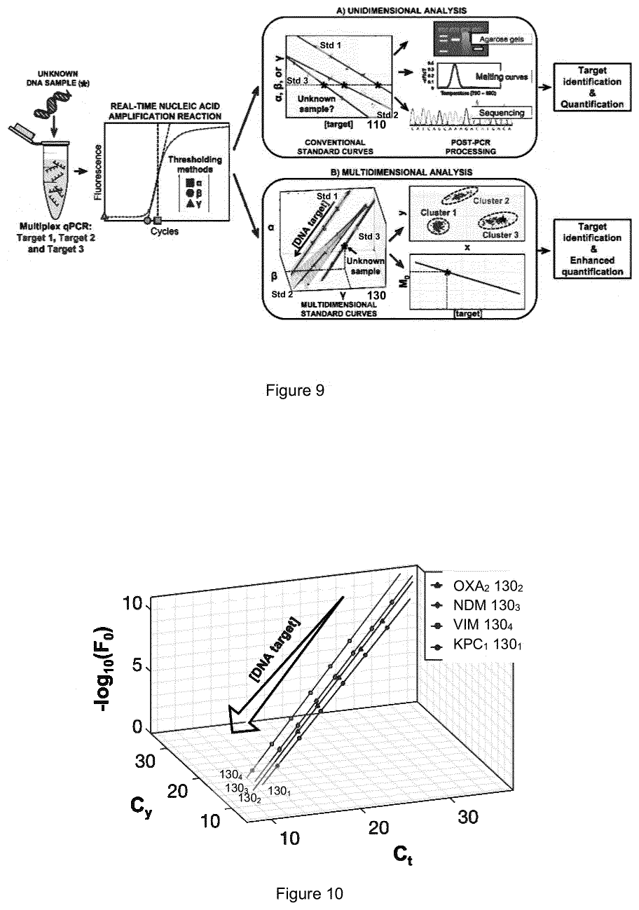

This example application of the disclosed method, as described with reference to FIGS. 9 to 12 and 16, describes the methodology disclosed herein, applied to generate multidimensional standard curves (MSC) for simultaneous DNA quantification, multiplex target discrimination and outlier detection using only amplification shapes. Herein, we propose the MSC for simultaneous nucleic acid quantification, outlier detection and single-channel multiplexing, without requiring melting curve analysis or any other post-PCR manipulation. The methodology disclosed herein combines multiple features of the amplification curve that are linear to the target concentration, such as Cₜ, F₀, and C_{y0}, to generate a characteristic fingerprint for each amplification curve. Then, the fingerprint is plotted in a multidimensional space to generate multivariate standard curves which provide enough information gain for simultaneous quantification, multiplexing and outlier detection. This method has been validated for the rapid screening of the four most clinically relevant carbapenemase genes (blaKPC, blaVIM, blaNDM and blaOXA-48) and has been shown to enhance quantification compared to the current state-of-the methods. The proposed method thus has the potential to deliver more comprehensive and actionable diagnostics, leading to improved patient care and reduced healthcare costs.

FIG. 9 is an Illustration of an example experimental workflow for single-channel multiplex quantitative PCR using unidimensional and multidimensional analysis approach. In this example, an unknown DNA sample is amplified by multiplex qPCR for targets 1, 2 and 3. Features such as a, β and γ are extracted from the amplification curve. It is important to stress that any number of targets and features could have been chosen.

In the example conventional uni-dimensional analysis shown at FIG. 9 (A), three conventional standard curves are generated through serial dilution of the known targets using a single feature. Given it is not possible to identify the target based on these standard curves, postPCR analysis are required for target identification and quantification. For example, threshold Cₜ is plotted against log 10 concentration of reference target1 and a regression line fitting the data is generated to construct the Standard1 (Std 1). Relative values for target abundance in the unknown sample are extrapolated from the unidimensional standard. However, in single-channel qPCR multiplexing assays, the presence of multiple standard curves prevents the identification and quantification of the target within the unknown sample, since it is not possible to extrapolate a single feature to a specific standard curve. Therefore, post-PCR analysis are required (such as agarose gels, melting curves or sequencing) for target identification and quantification.

In the multidimensional analysis (B) disclosed herein, multidimensional standard curves and the feature space are used to simultaneously quantify and discriminate a target of interest solely based on the amplification curve: eliminating the need for expensive and time consuming post-PCR manipulations. Similar to conventional standard curves, multidimensional standard curves are generated by using standard solutions with known concentrations under uniform experimental conditions. In this example, multiple features, a, β and γ, are extracted from each amplification curve and plotted against each other. Because each amplification curve has been reduced to three values, it can be represented as a single point in a 3D space (a greater or lesser number of dimensions can be used in embodiments). In this example, amplification curves from each concentration for a given target will thus generate three-dimensional clusters, which can be connected by high dimensional line fitting to generate the target-specific multidimensional standard curves 130. The multidimensional space where all the data points are contained is referred to as the feature space, and those data points can be projected to an arbitrary hyperplane orthogonal to the standard curves for target classification and outlier detection. Unknown samples can be confidently classified through the use of clustering techniques and enhanced quantification can be achieved by combining all the features into a unified feature called M₀. It is important to stress that any number of targets and features could have been chosen, a three-plex assay and three features have been selected in this example to illustrate the concept in a comprehensive manner.

Example Primers and Amplification Reaction Conditions

All oligonucleotides were synthesised by Integrated DNA Technologies (The Netherlands) with no additional purification. Primer names and sequences are shown in Table 3. Each amplification reaction was performed in 5 μL of final volume with 2.5 μL FastStart Essential DNA Green Master 2× concentrated (Roche Diagnostics, Germany), 1 μL PCR Grade water, 0.5 μL of 10× multiplex PCR primer mixture containing the four primer sets (5 μM each primer) and 1 μL of different concentrations of synthetic DNA or bacterial genomic DNA. PCR amplifications consisted of 10 min at 95.0 followed by 45 cycles at 95.0 for 20 sec, 68.0 for 45 sec and 72.0 for 30 sec. One melting cycle was performed at 95.0 for 10 sec, 65.0 for 60 sec and 97.0 for 1 sec (continuous reading from 65.0 to 97° C.) for validation of the specificity of the products. Each experimental condition was run 5 to 8 times loading the reactions into LightCycler 480 Multiwell Plates 96 (Roche Diagnostics, Germany) utilising a LightCycler 96 Real-Time PCR System (Roche Diagnostics, Germany).

TABLE 3
Primers used for the CPE multiplex qPCR assay.

| | | | Size |
|---|---|---|---|
| Target | Primer | Sequence | (bp) |
| bla_{OXA-48} | OXA-48-F | TGTTTTTGGTGGCATCGAT | 177 |
| | OXA-48-R | GTAAMRATGCTTGGTTCGC | |
| bla_{NDM} | NDM-F | TTGGCCTTGCTGTCCTTG | 82 |
| | NDM-R | ACACCAGTGACAATATCACCG | |
| bla_{VIM} | VIM-F | GTTTGGTCGCATATCGCAAC | 382 |
| | VIM-R | AATGCGCAGCACCAGGATAG | |
| bla_{KPC} | KPC-F | TCGCTAAACTCGAACAGG | 785 |
| | KPC-R | TTACTGCCCGTTGACGCCCAATCC | |

Sequences are given in the 5′ to 3′ direction. Size denotes PCR amplification products.

Synthetic and Genomic DNA Samples

Four gBlock® Gene fragments were purchased from Integrated DNA Technologies (The Netherlands) and resuspended in TE buffer to 10 ng/4 stock solutions (stored at −20° C.). The synthetic templates contained the DNA sequence from blaOXA, blaNDM, blaVIM and blaKPC genes required for the multiplex qPCR assay. Eleven pure cultures from clinical isolates were obtained (Table 4). One loop of colonies from each pure culture was suspended in 50 μL digestion buffer (Tris-HCl 10 mmol/L, EDTA 1 mmol/L, pH 8.0 containing 5 U/4 lysozime) and incubated at 37.0 for 30 min in a dry bath. 0.75 μL proteinase K at 20 μg/4 (Sigma) were subsequently added, and the solution was incubated at 56.0 for 30 min. After boiling for 10 min, the samples were centrifuged at 10,000×g for 5 min and the supernatant was transferred in a new tube and stored at −80.0 before use. Bacterial isolates included non-CPE producer Klebsiella pneumoniae and Escherichia coli as control strains.

TABLE 4
Samples used in this example.

| Sample ID | Bacterial Isolate | Carbapenemase genes |
|---|---|---|
| 1 | Klebsiella pneumoniae | bla_{OXA-48} |
| 2 | Escherichia coli | bla_{OXA-48} |
| 3 | Citrobacter Freundii | bla_{VIM} |
| 4 | Escherichia coli | bla_{NDM} |
| 5 | Klebsiella pneumoniae | bla_{OXA-48} |
| 6 | Klebsiella pneumoniae | bla_{NDM} |
| 7 | Pseudomonas aeruginosa | bla_{VIM} |
| 8 | Klebsiella pneumoniae | bla_{KPC} |
| 9 | Klebsiella pneumoniae | bla_{NDM} + bla_{KPC} |
| 10 | Klebsiella pneumoniae | non-producer |
| 11 | Escherichia coli | non-producer |

Example of the Disclosed Method

The data analysis for simultaneous quantification and multiplexing is achieved using the method previously described herein. Therefore, there are the following stages in data analysis: pre-processing 101, curve fitting 102, multi-feature extraction 113, high-dimensional line fitting 114, similarity measure (multidimensional analysis) 115 and dimensionality reduction 116.

Pre-processing 101: (optional) Background subtraction via baseline correction, in this example. This is accomplished by removing the mean of the first 5 fluorescent readings from each raw amplification curve.

Curve fitting 102: (optional) The 5-parameter sigmoid (Richard's curve) is fitted, in this example, to model the amplification curves:

$$F ( x ) = {F}_{b} + \frac{{F}_{max}}{{( 1 + {e}^{- ( x - c ) / b} )}^{d}}$$

where x is the cycle number, F(x) is the fluorescence at cycle x, F_{b} is the background fluorescence, Fₘₐₓ is the maximum fluorescence, c is the fractional cycle of the inflection point, b is related to the slope of the curve and d allows for an asymmetric shape (Richard's coefficient). The optimization algorithm used in this example to fit the curve to the data is the trust-region method and is based on the interior reflective Newton method. The lower and upper bounds for the 5 parameters, [F_{b}, Fₘₐₓ, c, b, d], are given in this example as: [−0.5, −0.5, 0, 0, 0.7] and [0.5, 0.5, 50, 100, 10] respectively.

Feature extraction 113: Three features are chosen in this example to construct the multidimensional standard curve: Cₜ, C_{y} and −log₁₀(F₀). The details of these features are not the focus of this disclosure. It will be appreciated that fewer, or a greater number of, features could be used in other examples.

Line fitting 114: The method of least squares is used for line fitting in this example, i.e. the first principal component in principal component analysis (PCA).

Similarity measure (multidimensional analysis) 115: The similarity measure used in this example is the Mahalanobis distance, d:

d={square root over ((p−P·(q₂−q₁)^{T}Σ⁻¹(p−P·(q₂−q₁))}

where p, P, q1 and q2 are given in equation (2), and Σ is the co-variance matrix of the training data used to approximate the distribution D.

Feature weights: In order to maximize quantification performance, different weights, a, can be assigned to each feature. In order to accomplish this, a simple optimization algorithm can be implemented. Equivalently, an error measure can be minimized. In this example, the error measure to minimize is the figure of merit described in the following subsection. The optimization algorithm is the Nelder-Mead simplex algorithm (32,33) with weights initialized to unity, i.e. beginning with no assumption on how good features are for quantification. This is a basic algorithm and only 20 iterations are used to find the weights so that there is little computational overhead.

Dimensionality reduction 116: Three dimensionality reduction techniques were used in order to compare their performance. The first 3 are simple projections onto each of the individual features, i.e. Cₜ, C_{y} and −log₁₀(F₀). The final method uses principal component regression to compute a feature termed M₀ using a vector

p=[Cₜ,C_{y},−log₁₀(F₀)]^{T}
where [·]^{T} denotes the transpose operator.

The general form for calculating M0 for an arbitrary number of features, as shown in equation (2) is given as:

$${M}_{0} = Φ ( p , q 1 , q 2 ) = \frac{{( p - q 1 )}^{T} ( q 2 - q 1 )}{{( q 2 - q 1 )}^{T} ( q 2 - q 1 )}$$

Where Φ computes the projection of the point p∈Rⁿ onto the multidimensional standard curve 130. The points q1,q2∈Rⁿ are any two distinct points that lie on the standard curve.

Evaluation of the standard curves is performed as described in the general disclosure above.

Results

In this example, it is shown that simultaneous robust quantification and multiplexing detection of blaOXA-48, blaNDM, blaVIM and blaKPC-lactamase genes in bacterial isolates can be achieved through analysing the fluorescent amplification curves in qPCR by using multidimensional standard curves. This section is broken into two parts: multiplexing and robust quantification. First, it is proven that single-channel multiplexing can be achieved, which is non-trivial and highly advantageous.

Target Discrimination Using Multidimensional Analysis

FIG. 11 shows four amplification curves and their respective derived melting curves specific for blaOXA, blaNDM, blaVIM and blaKPC genes. The four curves have been chosen to have similar Cₜ (19.4 0.5) thus each reaction has a different target DNA concentration. Using only this information, i.e. in a conventional technique, post-PCR processing such as melting curve analysis would be needed to differentiate the targets. The same argument applies when solely observing C_{y} and F₀.

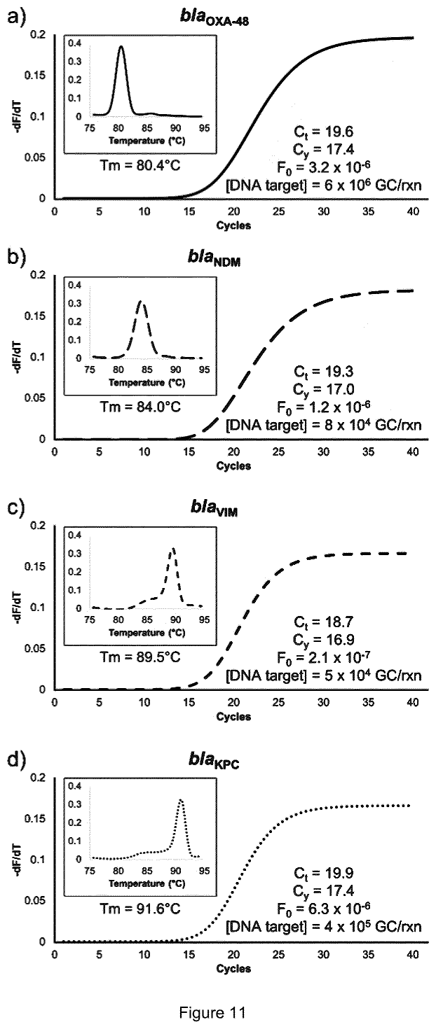

The multidimensional method disclosed herein shows that considering multiple features gives sufficient information gain in order to discriminate outliers from a specific target using a multidimensional standard curve 130. Taking advantage of this property, several multidimensional standard curves can be built in order to discriminate multiple specific targets. FIG. 10 shows the multidimensional standard curves 1301, 1302, 1303, 1304, constructed using a single primer fix for the four target genes using Cₜ, C_{y} and −log₁₀(F₀). It is visually observed that the 4 standards are sufficiently distant in multidimensional space in order to distinguish training samples. That is, an unknown DNA sample can be potentially classified as one of a number of specific targets (or an outlier) solely using the extracted features from amplification curves in a single channel.

In order to prove this, 11 samples given in Table 4 were tested against the multidimensional standards 1301, 1302, 1303, 1304. The similarity measure used to classify the unknown samples is the Mahalanobis distance, using a p-value of 0.01 as the threshold. In order to fully capture the position of the outliers in the feature space, it is convenient to view the feature space along the axis of the multidimensional standard curves 1301, 1302, 1303, 1304. Melting curves are provided in FIG. 11 to demonstrate that the real-time amplification curves belong to different qPCR products. Until the development of this methodology, it was not possible to associate amplification curve to a specific assay using a single-channel. Therefore, melting curves are used as a confirmation method.

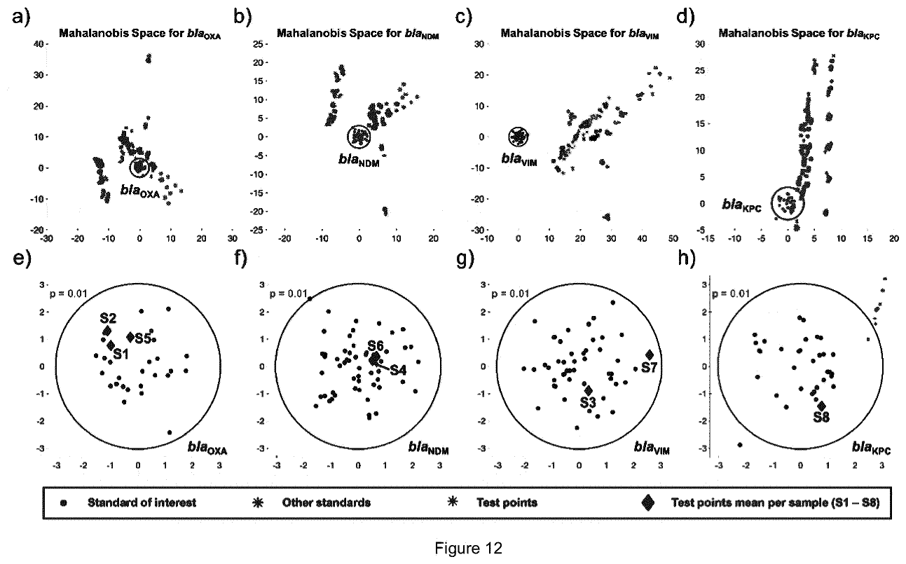

FIG. 12 shows the Mahalanobis space for the four standards in this example. This visualization is constructed by projecting all data points onto an arbitrary hyperplane orthogonal to each standard curve, as described in the general method disclosed above. The first observation is that the training points (synthetic DNA) from each standard are clustered together in its respective Mahalanobis space with a p-value<0.01. This corroborates the fact that there is sufficient information in the 3 chosen features to distinguish the 4 standard curves capturing the amplification reaction kinetics.

FIG. 12 uses the disclosed multidimensional analysis using the feature space for clustering and classification of unknown samples. As previously described, for this example arbitrary hyperplanes orthogonal to each multidimensional standard curve have been used to project all the data points, including the replicates for each concentration for the four multidimensional standards (training standard points) and eight unknown samples (test points). Circular callouts are magnified to visualise visualize the location of the samples relative to each standard of interest. The dark circular points within each magnified circular callout represent a standard of interest (5 to 8 replicates per each concentration), which is placed by default (0,0) at the centre of the Mahalanobis Space; dark grey asterisks represent the other standards; light grey asterisks represent the test points (3 replicates per sample); and the diamonds show the mean value for each sample. Each black circle corresponds to a p-value of 0.01.

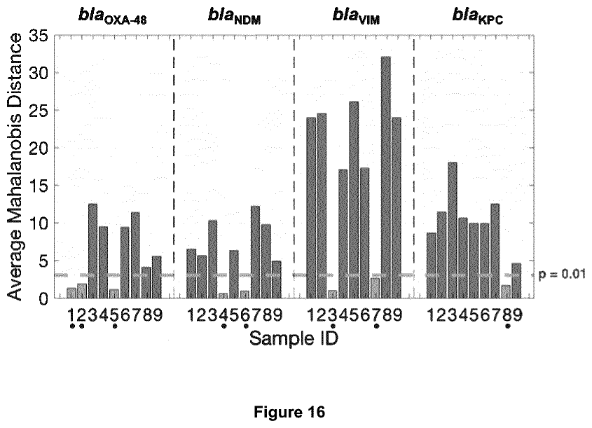

The second observation is that the mean of the test samples (bacterial isolates) which have a single resistance fall (samples 1-8) within the correct cluster (p-value<0.01) of training points. Melting curve analysis was used to validate the results, as provided in the Appendices. The results from testing can be succinctly captured within a bar chart as shown in FIG. 16. It is, however, important to the data in order to confirm that the Mahalanobis distance is a suitable similarity measure. When the training data points in the feature space are approximately normally distributed, then the distribution of the training data points in the Mahalanobis space is approximately circular—as seen in FIG. 6c. FIG. 16, in this example, shows average Mahalanobis distance from standard points to sample tests. The average distance between sample test points and the distribution of standard test points have been used to identify the presence of carbapenemase genes within the unknown samples. When the data is approximately normally distributed, the Mahalanobis Distance can be converted into a probability. Sample test points with an average distance relative to the standard of interest smaller than about 3.717 can be classified within this cluster (p-value<about 0.01). Samples 1, 2 and 5 were classified within blaOXA-48 cluster, samples 4 and 6 within blaNDM cluster, samples 3 and 7 within blaVIM cluster and sample 8 within blaKPC cluster. Sample 9 does not belong to any of the cluster (p-value>=about 0.01). After DNA amplification, melting curve analysis of the samples was also performed in order to determine the specificity of multiplex qPCR products. Melting curve analysis agrees well with sample classification based on the Mahalanobis distance.

It can be observed that using appropriate clustering techniques in each transformed space, it can be distinguished whether a point belongs to the target or not. Furthermore, if a probability is assigned to each data point then samples can be classified reliably to a given standard whilst simultaneously quantifying it. Given that the training data follow approximately a multivariate normal distribution, the Mahalanobis distance squared can provide a measure of probability.

Robust Quantification

Given that multiplexing has been established, quantification can be obtained using any conventional method such as the gold standard cycle threshold, Cₜ. However, as shown in the general method disclosed herein, enhanced quantification can be achieved using a feature, M₀, that combines all of the features for optimal absolute quantification. The measure of optimality in this study is a figure of merit that combines accuracy, precision, robustness and overall predictive power as shown in equation X. Table 5 shows the figure of merit for the 3 chosen features (Cₜ, C_{y} and −log₁₀(F₀)) and M₀ used in this example. The percentage improvement is also shown. It can be observed that quantification is always improved compared to the best single feature. The improvement is 30.69%, 14.39%, 2.12% and 35.00% for blaOXA-48, blaNDM, blaVIM and blaKPC respectively. This is a result of the multidimensional framework. It is further interesting to observe that amongst the conventional methods, there is no single method that performs the best for all the targets. Thus, M₀ is the most robust method in the sense that it will always be the best performing method.

TABLE 5
Figure of merit comparing conventional features
with M₀ for absolute quantification.

| | bla_{OXA-48} | bla_{NDM} | bla_{VIM} | bla_{KPC} |
|---|---|---|---|---|
| Cₜ | 2.71e+09 | 1.21e+08 | 2.45e+07 | 2.43e+09 |
| C_{y} | 2.12e+09 | 8.88e+07 | 9.74e+07 | 1.31e+09 |
| F₀* | 1.05e+10 | 1.98e+09 | 2.28e+09 | 2.17e+10 |
| M₀ | 1.47e+09 | 7.60e+07 | 2.40e+07 | 8.53e+08 |
| % Imp. | 30.69 | 14.39 | 2.12 | 35.00 |

% Imp. = Percentage improvement of M₀ over the next best method (both in bold)
*The figure of merit values is calculated using −log₁₀(F₀)

### Appendix A

Nucleotide sequence for synthetic double-stranded DNA ordered from Integrated DNA Technologies containing the lambda phage DNA target.

Forward lambda PCR primer in bold and reverse lambda primer in italics.

| | |
|---|---|
| gBlock | CAGGAACAGGGAATGCCCGTTCTGCGAGGCGGTGGCAAGGG |
| gene | TAATGAGGTGCTTTATGACTCTGCCGCCGTCATAAAATGGT |
| fragment | ATGCCGAAAGGGATGCTGAAATTGAGAACGAAAAGCTGCGC |
| | CGGGAGGTTGAAGAACTGCGGCAGGCCAGCGAGGCAGATCT |
| | CCAGCCAGGAACTATTGAGTACGAACGCCATCGACTTACGC |
| | GTGCGCAGGCCGACGCACAGGAACTGAAGAATGCCAG |

### Appendix B

Template preparation from bacterial isolates for real-time PCR assays.

One loop of colonies from the pure culture was suspended in 50 μL digestion buffer (Tris-HCl 10 mmol/L, EDTA 1 mmol/L, pH 8.0 containing 5 U/4 lysozime) and incubated at 37° C. for 30 min in a dry bath. 0.75 μL proteinase K at 20 μg/4 (Sigma) were subsequently added, and the solution was incubated at 56° C. for 30 min. After boiling for 10 min, the samples were centrifuged at 10,000×g for 5 min and the supernatant was transferred in a new tube and stored at −80 C before use.

### Appendix C

Experimental values for construction of lambda DNA standard.

242 bp of double-stranded DNA lambda phage was used to build molecule (gBlock gene fragment, IDT) containing the desired target sequence from the standard curves. Each condition run in octuplicate.

| | reaction | | | | | | | | |
|---|---|---|---|---|---|---|---|---|---|
| Copies | C_t | C_y | F_0 | FDM | Fb | Fmax | c | b | d |
| 1.00E+02 | 31.31642556 | 29.689285 | 1.953E−10 | 33.32652393 | 0.0015457 | 0.237249397 | 32.27105902 | 2.2666419 | 1.5930515 |
| | 30.85718263 | 29.241097 | 1.5809E−10 | 32.84914792 | 0.0014494 | 0.243261131 | 32.03282977 | 2.1674422 | 1.4573612 |
| | 30.38051354 | 28.778102 | 2.4672E−10 | 32.37117061 | 0.0015567 | 0.239087877 | 31.40173083 | 2.2147557 | 1.5491689 |
| | 31.01076063 | 29.348412 | 2.03E−10 | 32.92634828 | 0.0014582 | 0.262933142 | 31.91844747 | 2.2156504 | 1.5760168 |
| | 30.82737759 | 29.15149 | 2.0566E−10 | 32.77220907 | 0.0011658 | 0.245682733 | 31.68077043 | 2.2621916 | 1.6200704 |
| | 31.46299181 | 29.886402 | 9.3304E−11 | 33.41427582 | 0.0014616 | 0.24831291 | 32.45281216 | 2.1752586 | 1.5558153 |
| | 31.02750482 | 29.3932 | 1.6436E−10 | 33.00693613 | 0.0009706 | 0.238718542 | 32.34686963 | 2.1058819 | 1.3681226 |
| | 31.58078418 | 29.986653 | 1.1628E−10 | 33.5792156 | 0.0014866 | 0.245090098 | 32.66043256 | 2.1954679 | 1.5196663 |
| 1.00E+03 | 27.5284031 | 25.903247 | 1.0392E−09 | 29.44146907 | 0.001066 | 0.220418987 | 28.35971598 | 2.2159225 | 1.6293364 |
| | 27.66916052 | 26.056862 | 9.159E−10 | 29.57888844 | 0.0012113 | 0.253821736 | 28.57454043 | 2.1819157 | 1.5845582 |
| | 27.56642447 | 25.917012 | 1.2046E−09 | 29.46941702 | 0.0010075 | 0.249604593 | 28.35415241 | 2.2308444 | 1.6486048 |
| | 27.57336126 | 25.938243 | 1.2251E−09 | 29.47960135 | 0.0013148 | 0.255766778 | 28.28045923 | 2.2559653 | 1.7015554 |
| | 27.536951 | 25.90981 | 1.5509E−09 | 29.51280778 | 0.0012972 | 0.26232684 | 28.54902311 | 2.2115873 | 1.546182 |
| | 27.57360898 | 25.893945 | 1.9572E−09 | 29.49244838 | 0.0012449 | 0.277218703 | 28.1693003 | 2.3215693 | 1.7681555 |
| | 27.61091831 | 26.004337 | 9.0342E−10 | 29.52348965 | 0.0007348 | 0.25704513 | 28.64515394 | 2.1303722 | 1.5102756 |
| | 27.44180436 | 25.850647 | 1.4957E−09 | 29.46879316 | 0.0011955 | 0.243998447 | 28.75689668 | 2.1307049 | 1.3967011 |
| 1.00E+04 | 24.06984357 | 22.435534 | 8.1662E−09 | 26.00176569 | 0.0001948 | 0.175985083 | 25.34585343 | 2.0683532 | 1.3731647 |
| | 24.20374102 | 22.548889 | 9.8175E−09 | 26.06615692 | 0.000653 | 0.245890188 | 24.98188214 | 2.1967766 | 1.6381628 |
| | 24.21170567 | 22.528028 | 1.2964E−08 | 26.08908438 | 0.0010551 | 0.260040179 | 24.851171 | 2.2738706 | 1.7235878 |
| | 24.18620913 | 22.503267 | 1.4003E−08 | 26.07881565 | 0.0011238 | 0.268945989 | 24.89657201 | 2.264822 | 1.6853999 |
| | 24.19058629 | 22.486456 | 1.6537E−08 | 26.07577406 | 0.0011564 | 0.271623661 | 24.75818677 | 2.3139884 | 1.7672082 |
| | 24.26095613 | 22.525101 | 1.8405E−08 | 26.14064405 | 0.0009268 | 0.263626765 | 24.64592334 | 2.3768067 | 1.8755045 |
| | 24.37280071 | 22.649507 | 1.5585E−08 | 26.25781457 | 0.0009228 | 0.266626354 | 24.80666575 | 2.3601348 | 1.8493948 |
| | 24.22734488 | 22.576414 | 1.1968E−08 | 26.13897868 | 0.000968 | 0.265854062 | 25.14496267 | 2.1951626 | 1.5727428 |
| 1.00E+05 | 20.63429871 | 18.90862 | 9.2249E−08 | 22.43951121 | 0.0007144 | 0.213142097 | 20.8967991 | 2.3439163 | 1.9312687 |
| | 20.66751826 | 18.992227 | 7.0776E−08 | 22.46736597 | 0.0002674 | 0.23125111 | 21.21487621 | 2.2206573 | 1.7577201 |
| | 20.70957685 | 19.010783 | 7.2462E−08 | 22.47662304 | 0.0004681 | 0.233422197 | 21.00349467 | 2.2835078 | 1.9062089 |
| | 20.66725424 | 18.930487 | 1.0442E−07 | 22.48589535 | 0.0007851 | 0.238945789 | 20.97710635 | 2.34736 | 1.9017223 |
| | 20.61225857 | 18.943148 | 1.0621E−07 | 22.51055486 | 0.0008116 | 0.251415346 | 21.39089135 | 2.2368148 | 1.6496474 |
| | 20.6473748 | 18.97289 | 8.4147E−08 | 22.48108019 | 0.0005546 | 0.236007899 | 21.23331363 | 2.2416678 | 1.7447726 |
| | 20.71351121 | 18.954878 | 1.1928E−07 | 22.53086914 | 0.0006235 | 0.252754773 | 21.01011843 | 2.3583056 | 1.905699 |
| | 20.63017313 | 18.978005 | 9.8233E−08 | 22.51374731 | 0.0008541 | 0.24877384 | 21.36538533 | 2.2300263 | 1.6735623 |
| 1.00E+06 | 17.52039641 | 15.849225 | 5.8063E−07 | 19.30914223 | 0.0002711 | 0.233341053 | 17.98626328 | 2.2335487 | 1.8081003 |
| | 17.53211988 | 15.885981 | 5.6976E−07 | 19.35141128 | 0.0001535 | 0.233643726 | 18.23173271 | 2.172687 | 1.6742123 |
| | 17.55068349 | 15.868372 | 6.4324E−07 | 19.33767282 | 0.0004999 | 0.253644523 | 17.93107266 | 2.2662734 | 1.8601676 |
| | 17.54196046 | 15.830246 | 7.8548E−07 | 19.33374058 | 0.0006168 | 0.26356721 | 17.76996301 | 2.3305762 | 1.9561597 |
| | 17.50681431 | 15.844843 | 7.4948E−07 | 19.36656686 | 0.0005813 | 0.249012055 | 18.16594024 | 2.2343588 | 1.7114608 |
| | 17.52769391 | 15.874315 | 6.5335E−07 | 19.36004448 | 0.0004442 | 0.247523626 | 18.16934891 | 2.2100455 | 1.7138892 |
| | 17.51237224 | 15.856772 | 6.0967E−07 | 19.33029282 | 0.0002788 | 0.246961405 | 18.15911777 | 2.1948766 | 1.7050509 |
| | 17.54855322 | 15.881715 | 6.3777E−07 | 19.36201835 | 0.0002879 | 0.249542843 | 18.14635936 | 2.2122174 | 1.7324223 |
| 1.00E+07 | 13.96696278 | 12.20738 | 6.11E−06 | 15.6748737 | 0.0003483 | 0.229777492 | 14.201394 | 2.2824471 | 1.907074 |
| | 13.84637735 | 12.233504 | 5.81E−06 | 15.72979751 | 1.131E−05 | 0.218461699 | 15.04855666 | 2.0378743 | 1.3969481 |
| | 14.00744519 | 12.26807 | 7.3704E−06 | 15.71493378 | 0.0002928 | 0.249736247 | 14.21217722 | 2.2780935 | 1.9341256 |
| | 13.99563527 | 12.260033 | 8.0077E−06 | 15.7078218 | 0.0003488 | 0.262930563 | 14.14314769 | 2.2963335 | 1.9766022 |
| | 13.9949229 | 12.295078 | 6.1692E−06 | 15.74775577 | 0.0001653 | 0.257466087 | 14.58830608 | 2.1783029 | 1.7027967 |
| | 14.00779065 | 12.285854 | 7.8329E−06 | 15.75027197 | 0.0003001 | 0.270111228 | 14.47819476 | 2.2206618 | 1.7732907 |
| | 14.01237511 | 12.298749 | 7.0768E−06 | 15.7442183 | 3.722E−05 | 0.250274732 | 14.47482342 | 2.2058977 | 1.7779393 |
| | 14.01995332 | 12.307153 | 7.4742E−06 | 15.76709861 | 0.0002119 | 0.260476408 | 14.51591565 | 2.2108118 | 1.7610993 |
| 1.00E+08 | 10.46640035 | 8.7311252 | 6.1266E−05 | 12.15442454 | −1.668E−05 | 0.215403429 | 10.34233916 | 2.3421986 | 2.167704 |
| | 10.49143342 | 8.740428 | 7.8192E−05 | 12.16232834 | 5.078E−05 | 0.274393058 | 10.22732828 | 2.3732284 | 2.2599554 |
| | 10.4853575 | 8.7630979 | 6.7711E−05 | 12.19494802 | −7.463E−05 | 0.241039869 | 10.5111501 | 2.3127438 | 2.0710424 |
| | 10.50907176 | 8.7411068 | 8.1249E−05 | 12.18915375 | 3.412E−05 | 0.2711017 | 10.19485199 | 2.4019621 | 2.2939616 |
| | 10.48262252 | 8.7996293 | 7.1877E−05 | 12.23602001 | −0.000254 | 0.269959065 | 10.89191743 | 2.2186605 | 1.8327492 |
| | 10.49819678 | 8.7829293 | 7.0938E−05 | 12.19851884 | −8.684E−05 | 0.269025191 | 10.54834034 | 2.2949582 | 2.0524724 |
| | 10.4881275 | 8.7650576 | 6.5242E−05 | 12.20347798 | −0.0001102 | 0.243375819 | 10.63728067 | 2.2842266 | 1.9850768 |
| | 10.47827478 | 8.7521108 | 7.7043E−05 | 12.20427685 | −0.0001149 | 0.26981506 | 10.60905866 | 2.299649 | 2.0010639 |

### Appendix D

| | Concentration | | | | | | | |
|---|---|---|---|---|---|---|---|---|
| | Replicate | 1.00E+08 | 1.00E+07 | 1.00E+06 | 1.00E+05 | 1.00E+04 | 1.00E+03 | 1.00E+02 |
| Relative | 1 | 5.5555 | 0.5114 | 9.5157 | 10.7036 | 9.0197 | 5.7072 | 17.9332 |
| Error | 2 | 3.7877 | 7.921 | 10.2285 | 8.2501 | 0.3972 | 3.8695 | 11.8746 |
| (per trial) | 3 | 4.214 | 3.192 | 11.3459 | 5.2215 | 0.931 | 3.0301 | 54.3126 |
| | 4 | 2.5599 | 2.4175 | 10.8226 | 8.2693 | 0.7879 | 2.549 | 0.8628 |
| | 5 | 4.4065 | 2.3706 | 8.6827 | 12.3621 | 0.4907 | 5.0994 | 14.147 |
| | 6 | 3.3152 | 3.2146 | 9.9601 | 9.7313 | 4.1688 | 2.5319 | 25.6601 |
| | 7 | 4.0194 | 3.5135 | 9.0245 | 4.9426 | 11.1341 | 0.0169 | 0.2702 |
| | 8 | 4.7132 | 4.0055 | 11.2184 | 11.0122 | 1.9708 | 12.0674 | 31.3394 |
| Relative | | 4.071425 | 3.3932625 | 10.0998 | 8.8115875 | 3.612525 | 4.358925 | 19.549988 |
| Error (RE) | | | | | | | | |
| Coefficient of | | 2.0597 | 1.3814 | 0.2129 | 0.3398 | 0.5877 | 0.3721 | 1.8359 |
| Variation (CV) | | | | | | | | |
| Average RE | | 7.6996446 | | | | | | |
| Average CV | | 0.9699286 | | | | | | |
| Relative | 1 | 6.0839 | 2.8016 | 10.8614 | 14.2799 | 7.0406 | 4.3254 | 17.8873 |
| Error | 2 | 5.4233 | 1.0142 | 13.0343 | 8.0415 | 0.8037 | 5.8983 | 10.9427 |
| (per trial) | 3 | 3.8308 | 1.3031 | 12 | 6.7038 | 0.5954 | 3.3657 | 51.3925 |
| | 4 | 5.3753 | 0.7691 | 9.7182 | 12.6143 | 2.2818 | 1.9027 | 3.2301 |
| | 5 | 1.3151 | 3.0768 | 10.5987 | 11.661 | 3.4428 | 3.8667 | 17.8223 |
| | 6 | 2.4575 | 2.4747 | 12.3504 | 9.4534 | 0.7933 | 4.979 | 28.0663 |
| | 7 | 3.6943 | 3.3154 | 11.3119 | 10.7851 | 7.2838 | 2.5206 | 0.172 |
| | 8 | 4.5996 | 3.8594 | 12.7848 | 9.0781 | 2.6202 | 8.0757 | 32.7488 |
| Relative | | 4.097475 | 2.3267875 | 11.582463 | 10.327138 | 3.1077 | 4.3667625 | 20.28275 |
| Error (RE) | | | | | | | | |
| Coefficient of | | 3.7033 | 0.8395 | 0.2516 | 0.3105 | 0.4419 | 0.3704 | 1.8874 |
| Variation (CV) | | | | | | | | |
| Average RE | | 8.0130107 | | | | | | |
| Average CV | | 1.1149429 | | | | | | |
| Relative | 1 | 1.4026 | 14.5468 | 31.622 | 5.0244 | 29.5711 | 22.9036 | 28.2305 |
| Error | 2 | 31.744 | 19.0407 | 32.9947 | 28.5293 | 14.1826 | 32.6766 | 2.2095 |
| (per trial) | 3 | 12.8921 | 4.5039 | 23.6794 | 26.7005 | 15.6453 | 9.6682 | 64.7824 |
| | 4 | 37.279 | 14.229 | 5.4332 | 8.4892 | 25.6179 | 8.0132 | 33.6652 |
| | 5 | 20.3618 | 13.6581 | 10.0757 | 10.4786 | 50.1636 | 18.472 | 35.5455 |
| | 6 | 18.6748 | 11.5559 | 22.3921 | 13.9454 | 68.4436 | 52.0679 | 41.9572 |
| | 7 | 8.4809 | 0.0428 | 27.9459 | 25.1322 | 40.9121 | 33.6609 | 6.5612 |
| | 8 | 29.6678 | 6.0835 | 24.376 | 1.6024 | 6.1358 | 13.9507 | 26.4939 |
| Relative | | 20.062875 | 10.457588 | 22.314875 | 14.98775 | 31.334 | 23.926638 | 29.930675 |
| Error (RE) | | | | | | | | |
| Coefficient of | | 36.6827 | 4.7492 | 2.2954 | 2.6891 | 3.0691 | 2.4236 | 2.4413 |
| Variation (CV) | | | | | | | | |
| Average RE | | 21.8592 | | | | | | |
| Average CV | | 7.7643429 | | | | | | |
| Relative | 1 | 5.705 | 0.4168 | 9.9004 | 11.7059 | 8.4528 | 5.3121 | 17.9187 |
| Error | 2 | 4.2501 | 5.9139 | 11.0345 | 8.1891 | 0.5133 | 4.4508 | 11.609 |
| (per trial) | 3 | 4.1055 | 2.6596 | 11.5324 | 5.6384 | 0.4998 | 3.1246 | 53.4789 |
| | 4 | 3.352 | 1.9521 | 10.5105 | 9.4846 | 1.2103 | 2.3648 | 1.5299 |
| | 5 | 3.5206 | 2.5719 | 9.2304 | 12.1627 | 1.3211 | 4.7487 | 15.1786 |
| | 6 | 3.0717 | 3.0047 | 10.6452 | 9.6513 | 2.7844 | 3.2218 | 26.3515 |
| | 7 | 3.9273 | 3.4572 | 9.6801 | 6.5686 | 10.0568 | 0.7352 | 0.1447 |
| | 8 | 4.6818 | 3.9637 | 11.6661 | 10.4597 | 2.1552 | 10.9203 | 31.742 |
| Relative | | 4.07675 | 2.9924875 | 10.52495 | 9.2325375 | 3.3742125 | 4.3597875 | 19.744163 |
| Error (RE) | | | | | | | | |
| Coefficient of | | 1.9088 | 1.1545 | 0.189 | 0.2922 | 0.5385 | 0.3651 | 1.8493 |
| Variation (CV) | | | | | | | | |

| | |
|---|---|
| Average RE | 7.7578411 |
| Average CV | 0.8996286 |

### Appendix E

Experimental values for outlier detection experiment.

Genomic DNA extracted from pure bacterial cultures. All targets at 1.00E+05 gDNA copies per reaction. Each condition run in octuplicate.

| | C_t | C_y | F_0 | FDM | Fb | Fmax | c | b | d |
|---|---|---|---|---|---|---|---|---|---|
| blaOXA | 22.184597 | 20.167014 | 5.7403E−07 | 24.531545 | 0.001076391 | 0.164580823 | 22.373002 | 2.9831429 | 2.06180181 |
| | 21.637173 | 19.667219 | 9.90172E−07 | 23.993578 | 0.001648503 | 0.203299854 | 21.782282 | 2.9846035 | 2.0978247 |
| | 21.491952 | 19.518798 | 9.00681E−07 | 23.849382 | 0.001268261 | 0.17532464 | 21.760572 | 2.9495887 | 2.03027233 |
| | 21.61322 | 19.641975 | 9.05066E−07 | 23.980733 | 0.00141739 | 0.184051845 | 21.859178 | 2.9654512 | 2.04505358 |
| | 21.558481 | 19.572417 | 9.41045E−07 | 23.883479 | 0.001126655 | 0.19108247 | 21.752885 | 2.9426859 | 2.06273013 |
| | 21.432695 | 19.451669 | 1.03468E−06 | 23.754751 | 0.001405818 | 0.191631438 | 21.459003 | 2.9892505 | 2.15545337 |
| | 21.449389 | 19.45573 | 1.03521E−06 | 23.802708 | 0.001315638 | 0.183544088 | 21.654205 | 2.9742447 | 2.05930678 |
| | 21.738299 | 19.774574 | 9.46506E−07 | 24.156169 | 0.001591928 | 0.189081341 | 22.145616 | 2.9628589 | 1.97108731 |
| blaNDM | 18.440486 | 16.099814 | 2.41274E−06 | 20.200161 | 0.000983918 | 0.196155618 | 12.369387 | 3.705956 | 8.27321998 |
| | 18.373231 | 16.033338 | 2.36331E−06 | 20.062808 | 0.001027311 | 0.212207279 | 12.061295 | 3.6668073 | 8.86532079 |
| | 18.38343 | 16.046074 | 2.24386E−06 | 20.076827 | 0.001014981 | 0.207600865 | 12.165542 | 3.6605451 | 8.68182201 |
| | 18.373006 | 16.019493 | 2.42077E−06 | 20.067082 | 0.001015963 | 0.211300278 | 12.001019 | 3.6854133 | 8.92311641 |
| | 18.436916 | 16.050714 | 2.38439E−06 | 20.155224 | 0.000818466 | 0.202140048 | 11.986712 | 3.7302755 | 8.93331732 |
| | 18.361913 | 16.050321 | 2.25549E−06 | 20.021069 | 0.001146539 | 0.215579616 | 12.023506 | 3.6263808 | 9.07373755 |
| | 18.349523 | 16.040497 | 2.06663E−06 | 19.991541 | 0.000988449 | 0.213749704 | 12.088669 | 3.598508 | 8.9903557 |
| | 18.381255 | 16.048216 | 2.16587E−06 | 20.056119 | 0.000989473 | 0.20719115 | 12.087935 | 3.6474637 | 8.88693505 |
| blaKPC | 19.931159 | 17.557041 | 7.40553E−06 | 22.398002 | 0.00123536 | 0.201573788 | 18.069608 | 3.7383429 | 3.18304296 |
| | 18.841497 | 16.525453 | 8.88964E−06 | 21.112652 | 0.001268713 | 0.211374284 | 16.200533 | 3.6840082 | 3.79377903 |
| | 18.893634 | 16.521401 | 8.80035E−06 | 21.153714 | 0.001162442 | 0.207455538 | 16.120942 | 3.7291701 | 3.85576342 |
| | 18.979895 | 16.623867 | 8.86451E−06 | 21.244209 | 0.001289258 | 0.21675431 | 16.25445 | 3.7171291 | 3.82810173 |
| | 19.159447 | 16.794291 | 7.34809E−06 | 21.483275 | 0.001009587 | 0.191127882 | 16.761188 | 3.7103629 | 3.57039054 |
| | 18.635578 | 16.319774 | 9.08735E−06 | 20.856911 | 0.001173194 | 0.208564098 | 15.726234 | 3.6847675 | 4.02450539 |
| | 18.537681 | 16.242353 | 8.40449E−06 | 20.730546 | 0.000985954 | 0.206029409 | 15.965329 | 3.5893616 | 3.77195848 |
| | 19.01092 | 16.688042 | 8.74399E−06 | 21.350863 | 0.001752902 | 0.212295602 | 16.779842 | 3.6889083 | 3.45259322 |

### Appendix F

Melting curve analysis for lambda DNA standard experiment as shown in FIG. 15a: This figure shows average melting curves peaks for synthetic lambda DNA standard experiments using the 242 bp double-stranded DNA molecule (gBlock gene fragment ordered from IDT) using in-house lambda primers. Ten-fold dilution from 10⁸ to 10¹ copies per reaction were used in this experiment, 8-reactions per tested concentration. Average melting curve peak was 80.49° C. (SD=0.08° C.) for all positive reactions and no secondary melting event was observed at other annealing temperatures.

Melting curve analysis for outlier detection experiment, as shown In FIG. 15b: This figure shows average melting curves peaks of 80.66° C. (SD=0.07° C.) for blaOXA48, 83.97° C. (SD=0.10° C.) for blaNDM and 90.76° C. (SD=0.10° C.) for blaKPC. Octuplicate reactions per gDNA sample were performed, 10⁶ genomic copies per reaction. No secondary melting event was observed at other annealing temperatures. Specific primers sets were selected from Monteiro et al 2012.

Melting curve analysis for primer concentration variation experiment, as shown in FIG. 15c: This figure shows average melting curves peaks for primer concentration experiments using phage lambda DNA and in-house lambda primers. Observed average melting curve peaks for tested primer concentration are: 80.18° C. (SD=0.09° C.) for 25 nM; 80.10° C. (SD=0.09° C.) for 100 nM; 80.18° C. (SD=0.04° C.) for 175 nM; 80.13° C. (SD=0.11° C.) for 250 nM; 80.21° C. (SD=0.21° C.) for 325 nM; 80.34° C. (SD=0.06° C.) for 400 nM; 80.46° C. (SD=0.08° C.) for 475 nM; 80.50° C. (SD=0.09° C.) for 550 nM; 80.63° C. (SD=0.09° C.) for 625 nM; 80.66° C. (SD=0.07° C.) for 700 nM; 80.73° C. (SD=0.06° C.) for 775 nM; and 80.87° C. (SD=0.07° C.) for 850 nM. Octuplicate reactions per primer concentration were performed. No secondary melting event was observed at other annealing temperatures.

Melting curve analysis for temperature variation experiment, as shown in FIG. 15d: This figure shows average melting curves peaks for temperature variation experiments using phage lambda DNA and in-house primers. Observed average melting curve peaks for tested temperatures are: 80.53° C. (SD=0.10° C.) for 52.0° C.; 80.52° C. (SD=0.13° C.) for 53.0° C.; 80.48° C. (SD=0.03° C.) for 54.9° C.; 80.53° C. (SD=0.07° C.) for 57.3° C.; 80.53° C. (SD=0.06° C.) for 59.9° C.; 80.43° C. (SD=0.17° C.) for 62.7° C.; 80.51 (SD=0.09° C.) for 65.4° C.; 80.51° C. (SD=0.09° C.) for 67.8° C.; 80.47° C. (SD=0.13° C.) for 69.9° C.; 80.35° C. (SD=0.09° C.) for 71.3° C.; 80.35° C. (SD=0.08° C.) for 71.9° C.; and 80.36° C. (SD=0.08° C.) for 72.0° C. Octuplicate reactions per tested temperature were performed. No secondary melting event was observed at other annealing temperatures.

### Appendix G

Experimental values for temperature variation experiment.

Lambda DNA as target (NEB, Catalog #N3011S), 10⁶ genomic copies per reaction. Temperature in Celsius. Each experimental condition run in octuplicate.

| Temperature | | | | | | | | | |
|---|---|---|---|---|---|---|---|---|---|
| (C.) | C_t | C_y | F_0 | FDM | Fb | Fmax | c | b | d |
| 52.0 | 15.783935 | 14.000508 | 1.55488E−06 | 17.440158 | 0.000411898 | 0.192964539 | 15.289587 | 2.4433774 | 2.4112937 |
| | 15.804857 | 14.033471 | 1.89315E−06 | 17.483679 | 0.0006732 | 0.247744976 | 15.502315 | 2.4114709 | 2.2742291 |
| | 15.79978 | 14.03821 | 1.59158E−06 | 17.474217 | 0.000465606 | 0.217403044 | 15.500295 | 2.3991774 | 2.2767513 |
| | 15.804352 | 14.033296 | 1.81295E−06 | 17.481732 | 0.000607157 | 0.235163187 | 15.472146 | 2.4167565 | 2.2968117 |
| | 15.803049 | 14.078793 | 1.5945E−06 | 17.511336 | 0.000317869 | 0.237090536 | 15.868738 | 2.3091769 | 2.0367081 |
| | 15.826753 | 14.085307 | 1.67692E−06 | 17.530154 | 0.000306196 | 0.237757059 | 15.812609 | 2.3354947 | 2.0863359 |
| | 15.81489 | 14.080646 | 1.52504E−06 | 17.536369 | 0.00034473 | 0.213043702 | 15.906451 | 2.3195789 | 2.0191528 |
| | 15.801422 | 14.110176 | 1.86066E−06 | 17.587338 | 0.000624766 | 0.24959253 | 16.19632 | 2.2682106 | 1.8464534 |
| 53.0 | 15.783756 | 14.036759 | 1.75339E−06 | 17.51244 | 0.000542965 | 0.210274665 | 15.766498 | 2.3654171 | 2.0919814 |
| | 15.782208 | 14.069832 | 1.80398E−06 | 17.528443 | 0.000503098 | 0.24588013 | 15.993133 | 2.2971455 | 1.9510265 |
| | 15.733792 | 13.959388 | 1.79318E−06 | 17.435158 | 0.000507655 | 0.200213895 | 15.418971 | 2.433439 | 2.2899597 |
| | 15.809626 | 14.071409 | 1.84958E−06 | 17.535864 | 0.000485122 | 0.245359722 | 15.864825 | 2.3368829 | 2.0443339 |
| | 15.814632 | 14.10752 | 1.69329E−06 | 17.550297 | 0.000346816 | 0.246288476 | 16.088117 | 2.2655049 | 1.9067687 |
| | 15.801807 | 14.109773 | 1.87294E−06 | 17.573306 | 0.000412118 | 0.254941486 | 16.189361 | 2.2551735 | 1.8472082 |
| | 15.840818 | 14.141904 | 1.61799E−06 | 17.584614 | 0.000193756 | 0.237961742 | 16.176257 | 2.2477298 | 1.8711789 |
| | 15.853865 | 14.151697 | 1.69643E−06 | 17.599081 | 0.000390063 | 0.251723323 | 16.177498 | 2.2570534 | 1.8773108 |
| 54.9 | 15.777866 | 14.08241 | 1.80172E−06 | 17.556192 | 0.000552298 | 0.226402281 | 16.103436 | 2.2838398 | 1.8891037 |
| | 15.815425 | 14.112629 | 1.73328E−06 | 17.571321 | 0.000338212 | 0.235427101 | 16.147815 | 2.2632052 | 1.875692 |
| | 15.820974 | 14.110013 | 1.80078E−06 | 17.580637 | 0.000494747 | 0.235019334 | 16.127294 | 2.2809045 | 1.891138 |
| | 15.843556 | 14.09773 | 2.17244E−06 | 17.592322 | 0.000601985 | 0.260821782 | 15.941812 | 2.3492499 | 2.0189331 |
| | 15.835764 | 14.118157 | 1.88639E−06 | 17.600664 | 0.000561814 | 0.236997568 | 16.11294 | 2.2981456 | 1.9104878 |
| | 15.829143 | 14.141557 | 1.80642E−06 | 17.61557 | 0.000430129 | 0.244145984 | 16.296436 | 2.2424248 | 1.800856 |
| | 15.838398 | 14.139888 | 1.64604E−06 | 17.607043 | 0.000294028 | 0.226080377 | 16.282847 | 2.2383643 | 1.8068608 |
| | 15.85278 | 14.160443 | 1.70836E−06 | 17.630398 | 0.000346177 | 0.237741663 | 16.328997 | 2.2337551 | 1.7907004 |
| 57.3 | 15.865191 | 14.092738 | 2.09542E−06 | 17.55227 | 0.000575836 | 0.237189086 | 15.538376 | 2.423321 | 2.2957217 |
| | 15.870339 | 14.109584 | 1.92227E−06 | 17.595791 | 0.000327314 | 0.22724696 | 15.898163 | 2.3535158 | 2.0571384 |
| | 15.83962 | 14.125577 | 1.90172E−06 | 17.601159 | 0.000446355 | 0.242472342 | 16.142178 | 2.2840647 | 1.894141 |
| | 15.814527 | 14.083501 | 2.27092E−06 | 17.58854 | 0.000624598 | 0.251433752 | 15.981294 | 2.3439674 | 1.9851504 |
| | 15.819732 | 14.108317 | 2.19797E−06 | 17.594988 | 0.000536717 | 0.259154734 | 16.124972 | 2.2941286 | 1.8979483 |
| | 15.830771 | 14.138156 | 1.94007E−06 | 17.621419 | 0.000477352 | 0.245466744 | 16.296908 | 2.2497026 | 1.8017339 |
| | 15.946097 | 14.171494 | 2.28183E−06 | 17.674609 | 0.000436813 | 0.254464845 | 15.909083 | 2.3818163 | 2.0985613 |
| | 15.831945 | 14.160115 | 2.054E−06 | 17.659669 | 0.00052317 | 0.253851044 | 16.484193 | 2.213737 | 1.7006178 |
| 59.9 | 15.753405 | 14.080609 | 1.76192E−06 | 17.540423 | 0.00017342 | 0.222481034 | 16.302425 | 2.2066304 | 1.7524858 |
| | 15.750003 | 14.074339 | 2.14082E−06 | 17.560052 | 0.000438492 | 0.252701442 | 16.31395 | 2.2267045 | 1.7500029 |
| | 15.757588 | 14.051247 | 2.26899E−06 | 17.554087 | 0.000594209 | 0.250277784 | 16.099798 | 2.2996452 | 1.8821174 |
| | 15.764854 | 14.058139 | 2.3919E−06 | 17.567638 | 0.000645951 | 0.258824584 | 16.136109 | 2.2972262 | 1.8648029 |
| | 15.814978 | 14.069426 | 2.48267E−06 | 17.593731 | 0.000580873 | 0.254670668 | 15.966587 | 2.3589858 | 1.9932453 |
| | 15.879203 | 14.087656 | 2.60259E−06 | 17.597857 | 0.00054089 | 0.261752149 | 15.605021 | 2.4448332 | 2.2594503 |
| | 15.921625 | 14.067088 | 2.53466E−06 | 17.572301 | 0.000655506 | 0.243292841 | 15.048887 | 2.5669535 | 2.6725644 |
| | 15.764967 | 14.102083 | 2.06692E−06 | 17.584961 | 0.000359073 | 0.253072707 | 16.398317 | 2.2057633 | 1.7125347 |
| 62.7 | 15.710415 | 13.948334 | 2.7049E−06 | 17.468899 | 0.000657299 | 0.235723381 | 15.538056 | 2.4384511 | 2.2074364 |
| | 15.657231 | 13.963526 | 2.32732E−06 | 17.464442 | 0.000686134 | 0.246368329 | 16.024107 | 2.2963585 | 1.8724089 |
| | 15.472239 | 13.91966 | 2.02897E−06 | 17.493997 | 0.000182834 | 0.186840611 | 17.045263 | 1.9917114 | 1.2526996 |
| | 15.714849 | 13.955173 | 2.54954E−06 | 17.479944 | 0.000784383 | 0.234600611 | 15.623844 | 2.4243329 | 2.1503114 |
| | 15.558146 | 13.943207 | 2.11083E−06 | 17.473593 | 0.000393969 | 0.212966594 | 16.588133 | 2.1346473 | 1.5140744 |
| | 15.765534 | 13.97032 | 2.91797E−06 | 17.487797 | 0.000733704 | 0.268943657 | 15.368059 | 2.4826311 | 2.3486184 |
| | 15.686329 | 14.003103 | 2.02122E−06 | 17.452742 | 0.000292909 | 0.242723443 | 16.054852 | 2.250059 | 1.8612872 |
| | 15.566326 | 13.869838 | 2.56994E−06 | 17.427379 | 0.000609436 | 0.210929848 | 16.039563 | 2.3119921 | 1.8226077 |
| 65.4 | 15.711372 | 13.797399 | 3.31518E−06 | 17.32656 | 0.000945471 | 0.23429961 | 13.780388 | 2.7846095 | 3.5733009 |
| | 15.6508 | 13.837792 | 2.58103E−06 | 17.322058 | 0.000853753 | 0.247464387 | 14.864075 | 2.5442716 | 2.6276382 |
| | 15.652046 | 13.839469 | 2.54695E−06 | 17.317964 | 0.000842823 | 0.247776337 | 14.837514 | 2.5456037 | 2.649592 |
| | 15.647109 | 13.809628 | 2.76558E−06 | 17.277611 | 0.001086398 | 0.260860619 | 14.445205 | 2.6163111 | 2.9523309 |
| | 15.682054 | 13.813195 | 2.63557E−06 | 17.281038 | 0.000916751 | 0.241151267 | 14.163539 | 2.669374 | 3.2151577 |
| | 15.656517 | 13.855113 | 2.49564E−06 | 17.318541 | 0.000815569 | 0.25537706 | 14.891503 | 2.5243006 | 2.6155367 |
| | 15.666606 | 13.877673 | 2.13707E−06 | 17.318375 | 0.000570605 | 0.234068087 | 14.983657 | 2.4873591 | 2.5564845 |
| | 15.682703 | 13.807599 | 2.89895E−06 | 17.308865 | 0.000847116 | 0.231517227 | 14.176712 | 2.6915042 | 3.2018175 |
| 67.8 | 15.61232 | 13.657878 | 2.65111E−06 | 17.173961 | 0.000848666 | 0.193625261 | 13.243341 | 2.8415572 | 3.9878911 |
| | 15.628404 | 13.640697 | 2.89065E−06 | 17.091843 | 0.001062254 | 0.247574991 | 12.235314 | 2.9300251 | 5.2462024 |
| | 15.632787 | 13.6352 | 2.97481E−06 | 17.08065 | 0.001073452 | 0.24750623 | 11.956401 | 2.9594847 | 5.6489332 |
| | 15.648754 | 13.600293 | 3.32674E−06 | 17.09533 | 0.001103429 | 0.242606766 | 11.28228 | 3.0725673 | 6.6320877 |
| | 15.655327 | 13.614337 | 2.92825E−06 | 17.088866 | 0.000959156 | 0.240552565 | 11.542583 | 3.0259307 | 6.252104 |
| | 15.670936 | 13.637914 | 3.43835E−06 | 17.164501 | 0.00134229 | 0.24431706 | 11.857322 | 3.0436895 | 5.7182693 |
| | 15.660201 | 13.688983 | 2.51378E−06 | 17.090232 | 0.000730122 | 0.244492309 | 12.39629 | 2.8683766 | 5.1368767 |
| | 15.662898 | 13.64074 | 3.12695E−06 | 17.069309 | 0.001067181 | 0.266465286 | 11.363612 | 3.0111079 | 6.6517707 |
| 69.9 | 15.6185 | 13.475912 | 5.20487E−06 | 17.19083 | 0.000817738 | 0.190254531 | 10.961329 | 3.2695101 | 6.7216358 |
| | 15.666112 | 13.348746 | 6.3955E−06 | 17.183364 | 0.000538346 | 0.243956411 | 9.302752 | 3.4913096 | 9.556371 |
| | 15.641634 | 13.333641 | 6.32668E−06 | 17.177663 | 0.00079869 | 0.228744944 | 9.1716065 | 3.5178046 | 9.7363589 |
| | 15.652216 | 13.360986 | 6.13783E−06 | 17.17087 | 0.000818476 | 0.245852914 | 9.3821072 | 3.4739139 | 9.4128074 |
| | 15.634845 | 13.347265 | 6.85141E−06 | 17.169928 | 0.001118262 | 0.244161786 | 9.2186486 | 3.505683 | 9.661136 |
| | 15.720987 | 13.341859 | 6.81223E−06 | 17.268752 | 0.000410835 | 0.245029448 | 9.0144864 | 3.585372 | 9.9962123 |
| | 15.647469 | 13.28847 | 7.3982E−06 | 17.210854 | 0.000575725 | 0.23464528 | 9.0439486 | 3.5813217 | 9.7807556 |
| | 15.687821 | 13.282487 | 7.64982E−06 | 17.294227 | 0.00038036 | 0.213127259 | 8.9045888 | 3.660439 | 9.8944684 |
| 71.3 | 15.890969 | 13.273536 | 2.16213E−05 | 17.774284 | 0.000185537 | 0.217774717 | 8.4647003 | 4.0905694 | 9.7363369 |
| | 15.804655 | 13.256535 | 2.01449E−05 | 17.644579 | 0.000265866 | 0.225562601 | 8.4606055 | 3.9991377 | 9.939219 |
| | 15.852729 | 13.292714 | 2.06154E−05 | 17.698515 | 0.000234507 | 0.23361324 | 8.4519564 | 4.0157298 | 9.9999985 |
| | 15.741773 | 13.225643 | 1.8842E−05 | 17.510209 | 0.000240554 | 0.244571556 | 8.5185633 | 3.9050226 | 9.9999983 |
| | 15.770319 | 13.231264 | 1.88213E−05 | 17.551556 | 0.000176967 | 0.244200454 | 8.5465316 | 3.9307397 | 9.884063 |
| | 15.868443 | 13.27752 | 2.1811E−05 | 17.72262 | 0.000209429 | 0.234003224 | 8.4550455 | 4.0459543 | 9.8806485 |
| | 15.874488 | 13.291105 | 2.16696E−05 | 17.724317 | 0.00018921 | 0.230485597 | 8.4688663 | 4.0354532 | 9.9099011 |
| | 16.168851 | 13.515986 | 2.08598E−05 | 18.122609 | −0.000128971 | 0.230183252 | 8.671903 | 4.1681799 | 9.6537446 |
| 71.9 | 18.304142 | 15.506286 | 2.43764E−05 | 20.665879 | 0.000438688 | 0.197831129 | 10.390319 | 4.6714915 | 9.0216897 |
| | 16.555301 | 13.911871 | 3.13691 E−05 | 18.708473 | 0.000665642 | 0.214917431 | 8.8918873 | 4.3722854 | 9.4421539 |
| | 16.811302 | 14.100171 | 2.6775E−05 | 18.956754 | 0.000292373 | 0.212844897 | 9.1171896 | 4.4027666 | 9.3451672 |
| | 16.571792 | 13.884709 | 2.92527E−05 | 18.700104 | 0.000285385 | 0.213090095 | 8.8379299 | 4.37348 | 9.5352431 |
| | 17.243151 | 14.489413 | 2.3922E−05 | 19.470553 | 0.000321182 | 0.21761832 | 9.2626816 | 4.5258251 | 9.5397943 |
| | 17.126058 | 14.395191 | 2.57469E−05 | 19.3116 | 0.000365157 | 0.224613182 | 9.3279931 | 4.4612175 | 9.3733075 |
| | 16.750798 | 14.079232 | 2.83249E−05 | 18.87211 | 0.000319749 | 0.224628717 | 8.9639113 | 4.3609792 | 9.6989001 |
| | 17.441569 | 14.710791 | 2.97974E−05 | 19.67426 | 0.000319939 | 0.232089073 | 9.6418353 | 4.4978226 | 9.3045817 |
| 72.0 | 25.734232 | 9.8772105 | 0.003022624 | 39.070845 | −0.002337563 | 0.042891904 | 38.829427 | 13.27725 | 1.0183491 |
| | 17.558772 | 14.824757 | 3.02141E−05 | 19.848178 | 0.000664674 | 0.224121525 | 9.2513433 | 4.6021474 | 9.9999979 |
| | 18.514186 | 15.771497 | 2.57026E−05 | 20.908776 | 0.000612986 | 0.226536056 | 11.091544 | 4.6210695 | 8.3682959 |
| | 18.322103 | 15.539327 | 2.76408E−05 | 20.691904 | 0.000530817 | 0.220875769 | 10.443022 | 4.6659402 | 8.9937588 |
| | 18.203374 | 15.443548 | 3.03131E−05 | 20.54387 | 0.000708519 | 0.227027153 | 10.049948 | 4.6537644 | 9.5346442 |
| | 18.451965 | 15.68986 | 2.45523E−05 | 20.84121 | 0.000577347 | 0.213626345 | 11.023366 | 4.6313973 | 8.3298456 |
| | 19.002519 | 16.213708 | 2.03739E−05 | 21.462058 | 0.000841705 | 0.208634321 | 11.658729 | 4.7140728 | 8.0011715 |
| | 20.413631 | 17.613504 | 1.93675E−05 | 23.054795 | 0.000795235 | 0.215491878 | 14.00951 | 4.7746342 | 6.6488622 |

### Appendix H

Experimental values for primer concentration variation experiment.

Lambda DNA as target (NEB, Catalog #N3011S), 10⁶ genomic copies per reaction. Primer concentration in nanomolar (nM), ranging from 25 to 850 nM each primer. Each experimental condition run in octuplicate.

| Primer | | | | | | | | | |
|---|---|---|---|---|---|---|---|---|---|
| concentration | | | | | | | | | |
| (each) | C_T | C_Y | F_0 | FDM | Fb | Fmax | c | b | d |
| 25 nM | 15.145958 | 13.8492093 | 3.6849E−07 | 17.207822 | −8.6288E−05 | 0.141745576 | 17.5222418 | 1.50247792 | 0.811178243 |
| | 15.1517621 | 13.873423 | 3.49655E−07 | 17.2346777 | −0.0001063 | 0.143961141 | 17.5767913 | 1.48590876 | 0.794344008 |
| | 15.1536681 | 13.8596187 | 3.70405E−07 | 17.2285069 | −1.88404E−05 | 0.143766319 | 17.5501344 | 1.50472793 | 0.80755456 |
| | 15.1680123 | 13.8583264 | 3.96485E−07 | 17.2170655 | −2.49502E−05 | 0.147570801 | 17.4807022 | 1.53500576 | 0.842190022 |
| | 15.1734093 | 13.9085524 | 2.78524E−07 | 17.226003 | −0.000212764 | 0.143746665 | 17.5651491 | 1.46321427 | 0.793119342 |
| | 15.1737773 | 13.9091244 | 2.8896E−07 | 17.2366435 | −0.000189233 | 0.150611364 | 17.5926963 | 1.45814246 | 0.783344664 |
| | 15.1267965 | 13.8848675 | 2.47504E−07 | 17.2178991 | −0.00025667 | 0.136027928 | 17.6368366 | 1.41688209 | 0.744028731 |
| | 15.1938349 | 13.9329979 | 2.42269E−07 | 17.2211895 | −0.000328862 | 0.147282633 | 17.5633095 | 1.44409959 | 0.789063186 |
| 100 nM | 15.4743201 | 14.1056774 | 1.25253E−06 | 17.5680666 | −0.000108182 | 0.229823795 | 17.6509458 | 1.68710747 | 0.952062081 |
| | 15.491513 | 14.1194605 | 1.09086E−06 | 17.5663485 | −0.000132679 | 0.213142281 | 17.6279223 | 1.68996147 | 0.964220749 |
| | 15.4960455 | 14.1319236 | 1.02229E−06 | 17.5879813 | −9.6269E−05 | 0.205589388 | 17.6776268 | 1.68038883 | 0.948049955 |
| | 15.4995578 | 14.1298662 | 1.18927E−06 | 17.5908084 | −3.55262E−05 | 0.232439515 | 17.6580838 | 1.69563241 | 0.961101066 |
| | 15.4048179 | 14.1668319 | 5.61794E−07 | 17.59891 | −0.000409819 | 0.192794387 | 17.9994473 | 1.47914953 | 0.76277749 |
| | 15.5088087 | 14.1931725 | 8.47722E−07 | 17.6271901 | −0.000271589 | 0.216138689 | 17.8267447 | 1.60656939 | 0.883192909 |
| | 15.514133 | 14.2040118 | 7.81929E−07 | 17.621533 | −0.000339883 | 0.22177021 | 17.8403294 | 1.58637686 | 0.871166577 |
| | 15.5265775 | 14.187653 | 1.02297E−06 | 17.6208818 | −0.000461242 | 0.247224079 | 17.7891322 | 1.62171683 | 0.901452149 |
| 175 nM | 15.6315418 | 14.0581903 | 3.01395E−06 | 17.6349765 | 0.000346356 | 0.249327891 | 16.9883737 | 2.07137576 | 1.366374718 |
| | 15.604992 | 14.0904837 | 2.52589E−06 | 17.6476101 | 0.000338557 | 0.254511454 | 17.2690679 | 1.9555625 | 1.213576811 |
| | 15.4957889 | 14.0684971 | 2.17963E−06 | 17.6272763 | 7.31341E−05 | 0.219200784 | 17.5906094 | 1.79873034 | 1.020594106 |
| | 15.6516577 | 14.1056109 | 2.47887E−06 | 17.6242453 | 0.000169768 | 0.25991853 | 17.0732084 | 2.00260121 | 1.316742058 |
| | 15.649219 | 14.1577265 | 2.08667E−06 | 17.6607924 | 2.46857E−05 | 0.253000675 | 17.3445036 | 1.89755291 | 1.181379085 |
| | 15.6556913 | 14.172173 | 2.08224E−06 | 17.6822788 | 5.16601E−06 | 0.24831066 | 17.4242903 | 1.87564384 | 1.147455189 |
| | 15.6616211 | 14.1727802 | 2.02134E−06 | 17.6754689 | −0.000100112 | 0.249545687 | 17.4189448 | 1.8697559 | 1.147053657 |
| | 15.6703562 | 14.1806317 | 2.15082E−06 | 17.6799752 | −0.000147193 | 0.262927616 | 17.3833102 | 1.88490465 | 1.170451937 |
| 250 nM | 15.8130344 | 13.9765768 | 4.16285E−06 | 17.5391764 | 0.001424081 | 0.297060248 | 14.9390109 | 2.62681719 | 2.690841579 |
| | 15.7071735 | 14.0909686 | 3.01614E−06 | 17.6198044 | 0.000445073 | 0.305152931 | 16.7425308 | 2.12949183 | 1.509779795 |
| | 16.1095294 | 13.8895628 | 8.9738E−06 | 17.5911409 | 0.000533337 | 0.352428629 | 10.0506717 | 3.35994055 | 9.433120631 |
| | 15.7280053 | 14.0943659 | 3.22239E−06 | 17.6343625 | 0.000511014 | 0.309151087 | 16.6788903 | 2.16251832 | 1.555556084 |
| | 15.7108146 | 14.1212377 | 2.63958E−06 | 17.6471725 | 0.000219398 | 0.283679615 | 16.9318227 | 2.0682517 | 1.41322133 |
| | 15.7052591 | 14.1080701 | 2.76472E−06 | 17.6498052 | 0.000282678 | 0.274881135 | 16.9162934 | 2.08391973 | 1.421889458 |
| | 16.1612765 | 13.8695338 | 5.56141E−06 | 17.6089218 | −0.000110864 | 0.326945309 | 9.78829373 | 3.39645626 | 9.999995659 |
| | 15.731979 | 14.1366311 | 2.69941E−06 | 17.6714825 | 0.000183952 | 0.278439284 | 16.9698302 | 2.06740705 | 1.404087459 |
| 325 nM | 15.7401104 | 14.0565735 | 2.82869E−06 | 17.5437753 | 0.000416579 | 0.316230526 | 16.2264046 | 2.2471005 | 1.797242606 |
| | 15.7169376 | 14.0322236 | 2.93939E−06 | 17.5602273 | 0.000792158 | 0.296154441 | 16.2583934 | 2.26985261 | 1.774524249 |
| | 16.3665002 | 13.6046929 | 1.32101E−05 | 18.1361271 | −0.001267609 | 0.422860615 | 8.72388188 | 4.08769987 | 9.999922875 |
| | 15.9737041 | 13.9835667 | 3.6681E−06 | 17.4661942 | 0.001108011 | 0.331761371 | 13.3288185 | 2.84621995 | 4.278655269 |
| | 15.9053005 | 13.9182074 | 5.56265E−06 | 17.4357995 | 0.001110243 | 0.317675616 | 12.7220943 | 2.95101876 | 4.939749165 |
| | 16.526687 | 13.5361079 | 2.15717E−05 | 18.5419134 | −0.001676003 | 0.438671163 | 8.16628564 | 4.50607805 | 9.999999036 |
| | 16.8350211 | 14.3746987 | 7.45252E−05 | 19.5041854 | 0.005940712 | 0.5 | 8.42583887 | 4.81141408 | 9.999285537 |
| | 15.7539988 | 14.097548 | 2.56911E−06 | 17.5945752 | 0.000242161 | 0.287876299 | 16.4972942 | 2.18295526 | 1.653110174 |
| 400 nM | 15.7843216 | 14.0217578 | 3.07304E−06 | 17.5210042 | 0.00076205 | 0.31814086 | 15.6784251 | 2.40256167 | 2.153130221 |
| | 15.7759352 | 13.9887631 | 3.18918E−06 | 17.4845716 | 0.00104403 | 0.31469123 | 15.32814 | 2.48182652 | 2.384260337 |
| | 15.8424911 | 13.9151629 | 2.66182E−06 | 17.3157837 | 0.001301951 | 0.315525443 | 13.9146332 | 2.68092507 | 3.556041931 |
| | 15.8636156 | 13.9282349 | 3.05373E−06 | 17.3321166 | 0.001516086 | 0.334461884 | 13.114944 | 2.81375413 | 4.476183688 |
| | 15.8609134 | 13.9931065 | 4.1296E−06 | 17.5191461 | 0.001378028 | 0.335996356 | 14.6450622 | 2.65865008 | 2.94771782 |
| | 15.8532289 | 14.028269 | 3.35201E−06 | 17.5588227 | 0.000991783 | 0.300254548 | 15.2165278 | 2.54440403 | 2.510714048 |
| | 15.8030412 | 14.0715307 | 2.42391E−06 | 17.5431069 | 0.000451668 | 0.277270963 | 15.89353 | 2.33353875 | 2.027694239 |
| | 15.8321726 | 14.0259206 | 3.04128E−06 | 17.52028 | 0.000812486 | 0.307110594 | 15.3183306 | 2.48858456 | 2.422548256 |
| 475 nM | 15.8442318 | 14.0430434 | 3.59281E−06 | 17.5437875 | 0.000940519 | 0.330612804 | 15.3726447 | 2.48590562 | 2.394994714 |
| | 15.8100351 | 13.9810216 | 2.9357E−06 | 17.44898 | 0.000934967 | 0.312894264 | 14.956735 | 2.5401191 | 2.667529576 |
| | 15.8829792 | 13.8574661 | 3.27949E−06 | 17.2136466 | 0.001501705 | 0.320230972 | 11.7798342 | 2.93544852 | 6.366826908 |
| | 15.9262167 | 13.8260666 | 5.04283E−06 | 17.3092555 | 0.00148772 | 0.357348056 | 10.8127489 | 3.12857181 | 7.976571719 |
| | 15.9525575 | 13.8902111 | 3.80925E−06 | 17.3411096 | 0.001241348 | 0.322583722 | 11.3065212 | 3.05947338 | 7.188102211 |
| | 15.8331946 | 14.0005373 | 2.81477E−06 | 17.4711711 | 0.000971397 | 0.29608315 | 14.8811547 | 2.56390452 | 2.746107357 |
| | 15.8175528 | 14.0081504 | 2.83583E−06 | 17.4720849 | 0.000832971 | 0.309221679 | 15.1403524 | 2.50114901 | 2.540255118 |
| | 16.0075608 | 13.9019232 | 5.38133E−06 | 17.3754946 | 0.001067369 | 0.334224877 | 10.8538323 | 3.11746078 | 8.100930645 |
| 550 nM | 15.8452619 | 13.9475758 | 3.86232E−06 | 17.4494095 | 0.001709766 | 0.340788744 | 14.1409212 | 2.73119034 | 3.358089734 |
| | 15.8370919 | 13.9575684 | 3.23588E−06 | 17.4059236 | 0.001120151 | 0.341989433 | 14.288003 | 2.65507441 | 3.235958326 |
| | 15.807696 | 13.9276892 | 3.40921E−06 | 17.4018863 | 0.001445485 | 0.331559709 | 14.2524234 | 2.68196779 | 3.235910984 |
| | 16.4752848 | 13.3922632 | 1.42226E−05 | 18.4150517 | −0.00184696 | 0.448660925 | 8.01212998 | 4.51793152 | 9.999999908 |
| | 15.8188568 | 13.9619488 | 3.18888E−06 | 17.40679 | 0.001201447 | 0.333807142 | 14.4007757 | 2.63356272 | 3.131227176 |
| | 15.9957872 | 13.8814527 | 5.42134E−06 | 17.3486743 | 0.001186067 | 0.36101393 | 10.5930722 | 3.13384356 | 8.633864474 |
| | 15.9165474 | 13.7231024 | 3.28499E−06 | 17.3297147 | 0.000634414 | 0.237665499 | 10.1707985 | 3.26660482 | 8.949041879 |
| | 15.8559544 | 13.9818756 | 3.05229E−06 | 17.4064185 | 0.000926307 | 0.33540894 | 14.2727357 | 2.64106314 | 3.275672687 |
| 625 nM | 15.8439501 | 13.9420273 | 3.40586E−06 | 17.3795775 | 0.001459548 | 0.357072834 | 13.8569864 | 2.72426016 | 3.643865472 |
| | 15.8510216 | 13.9468584 | 3.19796E−06 | 17.3755997 | 0.001328242 | 0.351682432 | 13.9365687 | 2.70297314 | 3.569102495 |
| | 16.0080094 | 13.7289746 | 6.27365E−06 | 17.4166242 | 0.001734048 | 0.362140838 | 9.60965778 | 3.39386377 | 9.977356266 |
| | 16.1511375 | 13.6494581 | 7.07573E−06 | 17.6923678 | 0.00016789 | 0.376483544 | 9.23106157 | 3.67877389 | 9.974524929 |
| | 15.8436187 | 13.9328367 | 3.99541E−06 | 17.4294507 | 0.002246076 | 0.345433353 | 13.732565 | 2.80303033 | 3.739264484 |
| | 15.7417365 | 13.8663403 | 2.85258E−06 | 17.2751071 | 0.001125285 | 0.299912052 | 13.8610666 | 2.68624829 | 3.564174937 |
| | 15.8503246 | 13.9557518 | 3.22103E−06 | 17.3830955 | 0.001393541 | 0.34172873 | 13.8949855 | 2.71204993 | 3.618836541 |
| | 15.8669826 | 13.9498362 | 3.10553E−06 | 17.3913474 | 0.001200457 | 0.325758632 | 13.7572575 | 2.74322988 | 3.761239605 |
| 700 nM | 15.8608075 | 13.9160567 | 3.50379E−06 | 17.3490698 | 0.001719733 | 0.348843164 | 13.1248286 | 2.83584279 | 4.435273754 |
| | 15.8582798 | 13.9315279 | 3.09323E−06 | 17.3516097 | 0.001368461 | 0.331417955 | 13.4483357 | 2.77510103 | 4.081783345 |
| | 15.0951759 | 13.5156926 | 1.88588E−06 | 17.2989364 | 0.000343244 | 0.099787299 | 17.27168 | 1.91829347 | 1.014310062 |
| | 15.8756325 | 13.9369406 | 3.25955E−06 | 17.3460863 | 0.001435683 | 0.345140378 | 13.1830204 | 2.80727432 | 4.405952968 |
| | 15.8404242 | 13.8654723 | 2.99277E−06 | 17.2790795 | 0.001201768 | 0.296547875 | 12.555482 | 2.88652407 | 5.136803571 |
| | 15.8562441 | 13.9473039 | 2.8823E−06 | 17.3669864 | 0.001129594 | 0.318690055 | 13.7307682 | 2.72923398 | 3.78983288 |
| | 15.8560821 | 13.9565621 | 2.73804E−06 | 17.3518792 | 0.001148223 | 0.320922331 | 13.7556037 | 2.70765785 | 3.774193939 |
| | 16.0180444 | 13.7716982 | 3.84722E−06 | 17.4052861 | 0.00070932 | 0.36168169 | 9.76035884 | 3.32015043 | 9.999994983 |
| 775 nM | 15.8544834 | 13.9308664 | 3.40574E−06 | 17.3615794 | 0.001658629 | 0.347644487 | 13.4588202 | 2.78522328 | 4.060221298 |
| | 15.8299195 | 13.9353015 | 2.77109E−06 | 17.358099 | 0.001257333 | 0.310497613 | 13.8754887 | 2.7081569 | 3.618178243 |
| | 15.8750837 | 13.9087437 | 2.93367E−06 | 17.3405974 | 0.001301259 | 0.316334695 | 13.0586045 | 2.8388745 | 4.5192305 |
| | 15.891885 | 13.902658 | 2.99562E−06 | 17.3386217 | 0.00120054 | 0.273282456 | 12.3931913 | 2.93157948 | 5.402980843 |
| | 15.8021818 | 13.8932777 | 3.97778E−06 | 17.3661188 | 0.001499067 | 0.382711369 | 14.0691431 | 2.70578362 | 3.382083743 |
| | 15.8692725 | 13.9511666 | 2.91922E−06 | 17.3976853 | 0.001175221 | 0.32191222 | 13.8646051 | 2.72931057 | 3.649154465 |
| | 15.8365188 | 13.95976 | 2.80316E−06 | 17.3601749 | 0.001265026 | 0.320709959 | 13.9416906 | 2.68284899 | 3.575837096 |
| | 15.8610202 | 13.9092998 | 3.20031E−06 | 17.3274536 | 0.00168607 | 0.335577183 | 12.9114383 | 2.85388527 | 4.699093445 |
| 850 nM | 15.8501264 | 13.9168004 | 3.44445E−06 | 17.3530546 | 0.001969313 | 0.348307633 | 13.2827404 | 2.81918804 | 4.236720596 |
| | 15.8581302 | 13.9284998 | 3.14543E−06 | 17.3336737 | 0.001587476 | 0.34010094 | 13.2724064 | 2.79248391 | 4.281727501 |
| | 15.8719648 | 13.898582 | 3.06985E−06 | 17.3203039 | 0.001431707 | 0.32824721 | 12.7865029 | 2.86860658 | 4.85733024 |
| | 15.9182852 | 13.8879927 | 3.4518E−06 | 17.3076019 | 0.001715976 | 0.343108292 | 11.4469935 | 3.02700649 | 6.931713212 |
| | 15.8685823 | 13.9243945 | 3.02092E−06 | 17.3134109 | 0.001401267 | 0.351528391 | 13.0640446 | 2.80570684 | 4.547346754 |
| | 15.9164867 | 13.9125076 | 3.37301E−06 | 17.2414842 | 0.001562952 | 0.355938254 | 11.3955933 | 2.9571277 | 7.22019124 |
| | 15.8439028 | 14.0333633 | 3.28555E−06 | 17.4956065 | 0.001321306 | 0.342805434 | 15.0604366 | 2.52849793 | 2.619777826 |
| | 15.9104744 | 13.9529068 | 3.66779E−06 | 17.3859345 | 0.001536225 | 0.367091386 | 13.1831532 | 2.82861467 | 4.418538867 |

Advantages and technical effects of aspects and embodiments, including those mentioned above, will be apparent to a skilled person from the foregoing description and from the Figures.

It will be appreciated that the described methods can be carried out by one or more computers under control of one or more computer programs arranged to carry out said methods, said computer programs being stored in one or more memories and/or other kinds of computer-readable media.

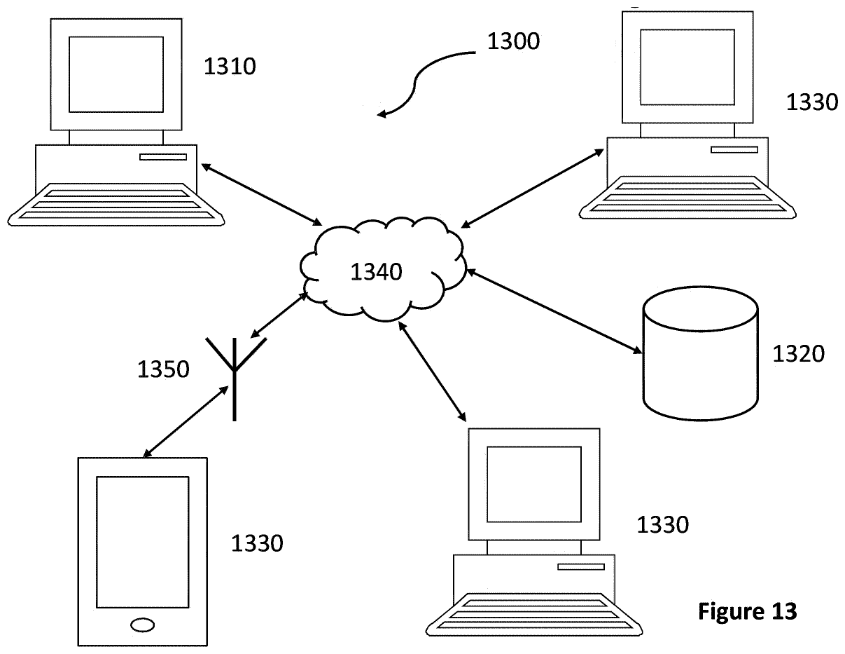

FIG. 13 shows an example of a computer system 1300 which can be used to implement the methods described herein, said computer system 1300 comprising one or more servers 1310, one or more databases 1320, and one or more computing devices 1330, said servers 1310, databases 1320 and computing devices 1330 communicatively coupled with each other by a computer network 1340. The network 1340 may comprise one or more of any kinds of computer network suitable for transmitting or communicating data, for example a local area network, a wide area network, a metropolitan area network, the internet, a wireless communications network 1350, a cable network, a digital broadcast network, a satellite communication network, a telephone network, etc. The computing devices 1330 may be mobile devices, personal computers, or other server computers. Data may also be communicated via a physical computer-readable medium (such as a memory stick, CD, DVD, BluRay disc, etc.), in which case all or part of the network may be omitted. Each of the one or more servers 1310 and/or computing devices 1330 may operate under control of one or more computer programs arranged to carry out all or a subset of method steps described with reference to any embodiment, thereby interacting with another of the one or more servers 1310 and/or computing devices 1330 so as to collectively carry out the described method steps in conjunction with the one or more databases 1320.

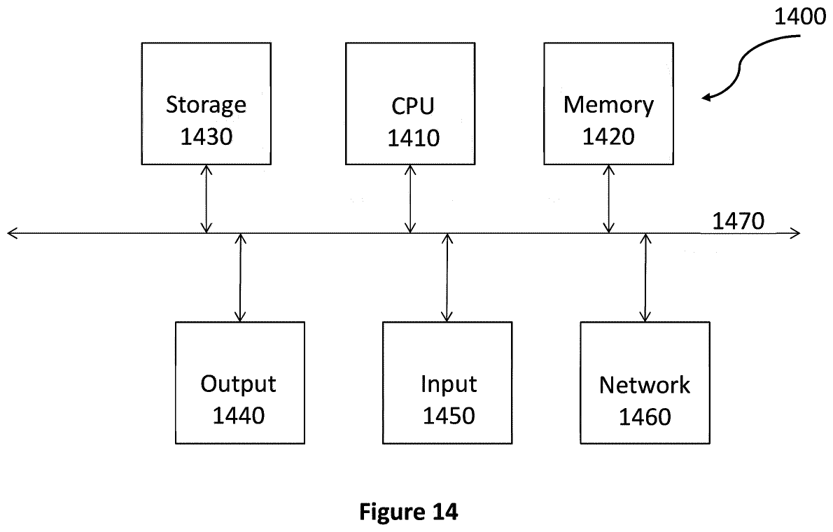

Referring to FIG. 14, each of the one or more servers 1310 and/or computing devices 1330 in FIG. 13 may comprise features as shown therein by way of example. The shown computer system 1400 comprises a processor 1410, memory 1420, computer-readable storage medium 1430, output interface 1440, input interface 1450 and network interface 1460, which can communicate with each other by virtue of one or more data buses 1470. It will be appreciated that one or more of these features may be omitted, depending on the required functionality of said system, and that other computer systems having fewer components or additional/alternative can be used instead, subject to the functionality required for implementing the described methods/systems.

The computer-readable storage medium may be any form of non-volatile and/or non-transitory data storage device such as a magnetic disk (such as a hard drive or a floppy disc) or optical disk (such as a CD-ROM, a DVD-ROM or a BluRay disc), or a memory device (e.g. a ROM, RAM, EEPROM, EPROM, Flash memory or portable/removable memory device) etc., and may store data, application program instructions according to one or more embodiments of the disclosure herein, and/or an operating system. The storage medium may be local to the processor, or may be accessed via a computer network or bus.

The processor may be any apparatus capable of carrying out method steps according to embodiments, and may for example comprise a single data processing unit or multiple data processing units operating in parallel or in cooperation with each other, or may be implemented as a programmable logic array, graphics processor, or digital signal processor, or a combination thereof.

The input interface is arranged to receive input from a user and provide it to the processor, and may comprise, for example, a mouse (or other pointing device), a keyboard and/or a touchscreen device.

The output interface optionally provides a visual, tactile and/or audible output to a user of the system, under control of the processor.

Finally, the network interface provides for the computer to send/receive data over one or more data communication networks.

Embodiments may be carried out on any suitable computing or data processing device, such as a server computer, personal computer, mobile smartphone, set top box, smart television, etc. Such a computing device may contain a suitable operating system such as UNIX, Windows® or Linux, for example.

It will be appreciated that the above-described partitioning of functionality can be altered without affecting the functionality of the methods and systems, or their advantages/technical effects. The above-described functional partitioning is presented as an example in order that the invention can be understood, and is thus conceptual rather than limiting, the invention being defined by the appended claims. The skilled person will also appreciate that the described method steps may be combined or carried out in a different order without affecting the advantages and technical effects resulting from the invention as defined in the claims.

It will be further appreciated that the described functionality can be implemented as hardware (for example, using field programmable gate arrays, ASICs or other hardware logic), firmware and/or software modules, or as a mixture of those modules. It will also be appreciated that, a computer-readable storage medium and/or a transmission medium (such as a communications signal, data broadcast, communications link between two or more computers, etc.), carrying a computer program arranged to implement one or more aspects of the invention, may embody aspects of the invention. The term “computer program,” as used herein, refers to a sequence of instructions designed for execution on a computer system, and may include source or object code, one or more functions, modules, executable applications, applets, servlets, libraries, and/or other instructions that are executable by a computer processor.

It will be further appreciated that the set of first data (training data) and second data (unknown sample data) can be obtained via the above-mentioned networked computer system components, such as by being retrieved from storage, being inputted by a user via an input device. Results data such as inlier/outlier determinations, and determined sample concentrations can also be stored using the aforementioned storage elements, and/or outputted to a display or other output device. The multidimensional standard curve 130 and/or the standard curve defined by the unidimensional function can also be stored using such storage elements. The aforementioned processor can process such stored and inputted data, as described herein, and store/output the results accordingly.

As will be appreciated by the skilled person, details of the above embodiment may be varied without departing from the scope of the present invention as defined by the appended claims. Many combinations, modifications, or alterations to the features of the above embodiments will be readily apparent to the skilled person and are intended to form part of the disclosure. Any of the features described specifically relating to one embodiment or example may be used in any other embodiment by making appropriate changes as apparent to the skilled person in the light of the above disclosure.

1. A method for quantifying a sample comprising a target nucleic acid, the method comprising:
obtaining a set of first real-time amplification data for each of a plurality of target concentrations;
extracting a plurality of N features from the set of first data, wherein each feature relates the set of first data to the concentration of the target; and
fitting a line to a plurality of points defined in an N-dimensional space by the features, each point relating to one of the plurality of target concentrations, wherein the line defines a multidimensional standard curve specific to the nucleic acid target which can be used for quantification target concentration.

2. The method of claim 1, further comprising:
obtaining second real-time amplification data relating to an unknown sample;
extracting a corresponding plurality of N features from the second data; and
calculating a distance measure between the line in N-dimensional space and a point defined in N-dimensional space by the corresponding plurality of N features.

3. The method of claim 2, further comprising computing a similarity measure between amplification curves from the distance measure, and optionally further comprising identifying outliers or classifying targets from the similarity measure.

4. The method of claim 1, wherein each feature is different to each of the other features, and optionally wherein each feature is linearly related to the concentration of the target, and optionally wherein one or more of the features comprises one of Cₜ, C_{y} and −log₁₀(F₀).

5. The method of claim 1, further comprising mapping the line in N-dimensional space to a unidimensional function, M₀, which is related to target concentration, and optionally wherein the unidimensional function is linearly related to target concentration, and/or optionally wherein the unidimensional function defines a standard curve for quantifying target concentration.

6. The method of claim 5, wherein the mapping is performed using a dimensionality reduction technique, and optionally wherein the dimensionality reduction technique comprises at least one of: principal component analysis; random sample consensus; partial-least squares regression; and projecting onto a single feature.

7. The method of claim 5, wherein the mapping comprises applying a respective scalar feature weight to each of the features, and optionally wherein the respective feature weights are determined by an optimization algorithm which optimizes an objective function, and optionally wherein the objective function is arranged for optimization of quantization performance.

8. The method of claim 2, wherein calculating the distance measure comprises projecting the point in N-dimensional space onto a plane which is normal to the line in N-dimensional space, and optionally wherein calculating the distance measure further comprises calculating, based on the projected point, a Euclidean distance and/or a Mahalanobis distance.

9. The method of claim 8, further comprising calculating a similarity measure based on the distance measure, and optionally wherein calculating a similarity measure comprises applying a threshold to the similarity measure.

10. The method of claim 9, further comprising determining whether the point in N-dimensional space is an inlier or an outlier based on the similarity measure.

11. The method of claim 10, comprising: if the point in N-dimensional space is determined to be an outlier then excluding the point from training data upon which the step of fitting a line to a plurality of points defined in N-dimensional space is based, and if the point in N-dimensional space is not determined to be an outlier then re-fitting the line in N-dimensional space based additionally on the point in N-dimensional space.

12. The method of claim 2, further comprising determining a target concentration based on the multidimensional standard curve, and optionally further based on the distance measure.

13. The method of claim 12, further including displaying the target concentration on a display.

14. The method of claim 1, wherein the method further comprises a step of fitting a curve to the set of first data, wherein the feature extraction is based on the curve-fitted first data, and optionally wherein the curve fitting is performed using one or more of a 5-parameter sigmoid, an exponential model, and linear interpolation, and optionally wherein the set of first data relating to the melting temperatures is pre-processed, and the curve fitting is carried out on the processed set of first data, and optionally wherein the pre-processing comprises one or more of: subtracting a baseline; and normalization.

15. The method of claim 1, wherein the data relating to the melting temperature is derived from one or more physical measurements taken versus sample temperature, and optionally wherein the one or more physical measurements comprise fluorescence readings.

16. The method of claim 1, used for single-channel multiplexing without post-PCR manipulations.

17. The method of claim 1, implemented using at least one processor and/or using at least one integrated circuit.

18. A system comprising at least one processor and/or at least one integrated circuit, the system arranged to carry out a method according to claim 1.

19. A computer program comprising instructions which, when executed by one or more processors, cause the one or more processors to perform a method according to claim 1.

20. A computer-readable medium storing instructions which when executed by at least one processor, cause the at least one processor to carry out a method according to claim 1.

21. The method of claim 1, used for detection of genomic material.

22. The method of claim 21, wherein the genomic material comprises one or more pathogens.

23. A method for diagnosis of an infection by detection of one or more pathogens according to the method of claim 1.

24. A method for point-of-care diagnosis of an infectious disease by detection of one or more pathogens according to the method of claim 1.

25. The method of claim 22, wherein the pathogens comprise one more carbapenemase-producing enterobacteria, and optionally wherein the pathogens comprise one or more carbapenemase genes from the set comprising blaOXA-48, blaVIM, blaNDM and blaKPC

26. The method of claim 5, further comprising determining a target concentration based on the unidimensional function which defines the standard curve.
